(19) United States
(12) Patent Application Publication
(10) Pub. No.: US 2012/0196895 A1
(43) Pub. Date: Aug. 2, 2012

(54) NOVEL FORMS OF EPERISONE

(75) Inventors: Isabel Kalofonos, Cambridge, MA (US); G. Patrick Stahly, West Lafayette, IN (US); William Martin-Doyle, Cambridge, MA (US); Dimitris Kalofonos, Cambridge, MA (US); Jeffrey S. Stults, West Lafayette, IN (US); Jason A. Hanko, West Lafayette, IN (US)

(73) Assignee: Bionevia Pharmaceuticals, Inc., Cambridge, MA (US)

(21) Appl. No.: 13/144,292

(22) PCT Filed: Jan. 11, 2010
(86) PCT No.: PCT/US10/20614

§ 371 Date: Apr. 9, 2012

Related U.S. Application Data
(60) Provisional application No. 61/143,701, filed on Jan. 9, 2009.

(51) Int. Cl. A61K 31/4453; A61P 21/02; A61P 29/00; A61P 25/14; A61P 25/00; C07D 295/108; A61P 21/00

(52) U.S. Cl. 514/317; 546/237

(57) ABSTRACT

The invention relates to novel crystalline forms of (2RS)-1-(4-ethylphenyl)-2-methyl-3-piperidin-1-yl-propan-1-one. The preparation and characterization of the novel crystalline forms according to various embodiments of the invention is described. The invention also relates to pharmaceutical compositions containing the novel crystalline forms, which are useful to treat and/or prevent various conditions such as pathological muscle contracture, myotonic conditions, and spastic paralysis or spasticity caused by various neurologic conditions, and are also useful for the treatment and/or prevention of various types of pain and pathological muscle tension.

### CROSS REFERENCE TO RELATED APPLICATIONS

This application claims priority under 35 U.S.C. §119 to U.S. Provisional Application 61/143,701, filed Jan. 9, 2009, which is incorporated herein by reference.

### TECHNICAL FIELD

The invention relates to novel forms of (2RS)-1-(4-ethylphenyl)-2-methyl-3-piperidin-1yl-propan-1one, processes for making those novel forms, pharmaceutical compositions comprising those novel forms, and methods of treating and/or preventing various conditions by administering those novel forms.

### BACKGROUND

The compound (2RS)-1-(4-ethylphenyl)-2-methyl-3-piperidin-1-yl-propan-1-one (shown below), referred to herein by its common name “eperisone,” is a known active pharmaceutical ingredient (API) having beneficial therapeutic activity, for example as a muscle relaxant and spasmolytic, and is useful in treating various conditions including pathological muscle contracture resulting from a variety of underlying musculoskeletal and neurologic conditions:

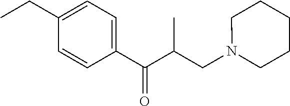

Racemic eperisone hydrochloride has a positive indication for the improvement of myotonic conditions caused by neck-shoulder-arm syndrome, scapulohumeral periarthritis, and low back pain, and for spastic paralysis or spasticity caused by various neurologic conditions, and is also useful for the treatment of various types of pain and pathological muscle tension. The preparation and pharmacologic activity of racemic eperisone hydrochloride is described for example in U.S. Pat. No. 3,995,047. Therapeutic activity in various conditions has been demonstrated in the clinical literature, for example in Bose K., Methods Find Exp Clin Pharmacol (1999) 21:209-13; Hanai K. et al., Jpn J Clin Exp Med (1983) 60:2049-2053; Hirohata K. et al., J New Remed Clin (1988) 37:200; Iwasaki T. et al., Nippon Ganka Gakkai Zasshi (1987) 91:740-6; Iwase S. et al., Funct Neurol. (1992) 7:459-70; Kobayashi Y. et al., Dig Dis Sci. (1992) 37:1145-6; Kuroiwa Y. et al., Jpn J Clin Exp Med (1980) 57:4033-4038; Kuroiwa Y. et al., Clin.Eval. (1981) 9:391-419; Mano T. et al., No To Shinkei (1981) 33:237-41; Mizuno K. et al., Prog Med (1991) 11:99-112; Murayama K. et al., Hinyokika Kiyo (1984) 30:403-8; Nakahara S. et al., Prog Med (1986) 6:11; Takayasu et al, Oncology (1989) 46(1): 58-60; and Nisijima K. et al., Acta Psychiatr Scand (1998) 98:341-3; U.S. Pat. No. 5,002,958; WO2004/089352; and U.S. Patent Application No. 20060004050.

Although therapeutic efficacy is a primary concern for a therapeutic agent, such as eperisone, the salt and solid-state form (e.g. crystalline or amorphous forms) of a drug candidate can be important to its pharmacological properties and to its development as a viable API. For example, each salt or each crystalline form of a drug candidate can have different solid-state (physical and chemical) properties. The differences in physical properties exhibited by a particular solid form of an API, such as a cocrystal, salt, or polymorph of the original compound, can affect pharmaceutical parameters of the API. For example, storage stability, compressibility and density, all of which can be important in formulation and product manufacturing, and solubility and dissolution rates, which may be important factors in determining bioavailability, may be affected. Because these physical properties are often influenced by the solid-state form of the API, they can significantly impact a number of factors, including the selection of a compound as an API, the ultimate pharmaceutical dosage form, the optimization of manufacturing processes, and absorption in the body. Moreover, finding the most adequate form for further drug development can reduce the time and the cost of that development.

Obtaining pure crystalline forms, then, is extremely useful in drug development. It may permit better characterization of the drug candidate's chemical and physical properties. For example, crystalline forms often have better chemical and physical properties than amorphous forms. As a further example, a crystalline form may possess more favorable pharmacology than an amorphous form, or may be easier to process. It may also have better storage stability.

One such physical property which can affect processability is the flowability of the solid, before and after milling. Flowability affects the ease with which the material is handled during processing into a pharmaceutical composition. When particles of the powdered compound do not flow past each other easily, a formulation specialist must take that fact into account in developing a tablet or capsule formulation, which may necessitate the use of additional components such as glidants, including colloidal silicon dioxide, talc, starch, or tribasic calcium phosphate.

Another solid state property of a pharmaceutical compound that may be important is its dissolution rate in aqueous fluid. The rate of dissolution of an active ingredient in a patient's stomach fluid may have therapeutic consequences since it can impact the rate at which an orally administered active ingredient may reach the patient's bloodstream.

Another solid state property of a pharmaceutical compound that may be important is its thermal behavior, including its melting point. The melting point of the solid form of a drug is optionally high enough to avoid melting or plastic deformation during standard processing operations, as well as concretion of the drug by plastic deformation on storage (See, e.g., Gould, P. L. Int. J. Pharmaceutics 1986 33 201-217). It may be desirable in some cases for a solid form to melt above about 100° C. For example, melting point categories used by one pharmaceutical company are, in order of preference, +(mp>120° C.), 0 (mp 80-120° C.), and −(mp<80° C.) (Balbach, S.; Korn, C. Int. J. Pharmaceutics 2004 275 1-12).

Active drug molecules may be made into pharmaceutically acceptable salts for therapeutic administration to the patient. Crystalline salts of a drug may offer advantages over the free form of the compound, such as improved solubility, stability, processing improvements, etc., and different crystalline salt forms may offer greater or lesser advantages over one another. However, crystalline salt formation is not predictable, and in fact is not always possible. Moreover, there is no way to predict the properties of a particular crystalline salt of a compound until it is formed. As such, finding the right conditions to obtain a particular crystalline salt form of a compound, with pharmaceutically acceptable properties, can take significant time and effort.

A crystalline form of a compound, a crystalline salt of the compound, or a cocrystal containing the compound or its salt form generally possesses distinct crystallographic and spectroscopic properties when compared to other crystalline forms having the same chemical composition. Crystallographic and spectroscopic properties of a particular form may be measured by XRPD, single crystal X-ray crystallography, solid state NMR spectroscopy, e.g. ¹³C CP/MAS NMR, or Raman spectroscopy, among other techniques. A particular crystalline form of a compound, of its salt, or of a cocrystal, often also exhibits distinct thermal behavior. Thermal behavior can be measured in the laboratory by such techniques as, for example, capillary melting point, TGA, and DSC.

Many organic compounds can exist as optically active forms, i.e., they have the ability to rotate the plane of plane-polarized light. In describing an optically active compound the prefixes R- and S-, and D- and L-, are used to denote the absolute configuration of the molecule about its chiral center(s). The prefixes d- and 1-, or (+)- or (−)-, designate the sign of rotation of plane-polarized light by the compound, with 1- or (-)- meaning that the compound is levorotatory. In contrast, a compound prefixed with d- or (+)- is dextrorotatory. There is no correlation between nomenclature for the absolute stereochemistry and for the rotation of light by an enantiomer. By way of example, D-lactic acid is the same as (−)-lactic acid, and L-lactic acid is the same as (+)-lactic acid. For a given chemical structure, each of a pair of enantiomers is identical except that they are non-superimposable mirror images of one another. In general, enantiomers have identical properties in a symmetrical environment, although their properties may differ in an unsymmetrical environment. A mixture of enantiomers is often called an enantiomeric, or racemic, mixture, or a racemate.

Currently, eperisone is available only as a racemic mixture of enantiomers of the hydrochloride salt, (+)- and (−)- in a 1:1 ratio, and reference herein to the generic name “eperisone” refers to this enantiomeric, or racemic, mixture. Racemic eperisone hydrochloride is commercially sold under the trade name MYONAL. Administration of racemic eperisone hydrochloride, however, can result in certain undesirable side effects such as, for example, insomnia, headache, nausea and vomiting, anorexia, abdominal pain, diarrhea, constipation, urinary retention, and/or incontinence, at least some of which may be avoided by the use of a different racemic salt form of the compound.

In the following description, various aspects and embodiments of the invention will become evident. In its broadest sense, the invention could be practiced without having one or more features of these aspects and embodiments. Further, these aspects and embodiments are exemplary. Additional objects and advantages of the invention will be set forth in part in the description which follows, and in part will be obvious from the description, or may be learned by practicing of the invention. The objects and advantages of the invention will be realized and attained by means of the elements and combinations particularly pointed out in the appended claims.

### SUMMARY

In accordance with various embodiments of the invention and after extensive experimentation, the inventors have discovered novel crystalline salt forms of eperisone, including crystalline racemic eperisone fumarate, crystalline racemic eperisone maleate, crystalline racemic eperisone mesylate, and crystalline racemic eperisone succinate.

The invention in various embodiments also relates to processes of preparing those crystalline salt forms of eperisone, pharmaceutical compositions containing them, and their use in the treatment and/or prevention of various conditions including, for example, myotonic conditions, pain, and pathological muscle tension, as well as improving blood flow.

As used herein, the term “XRPD” refers to x-ray powder diffraction. The XRPD data disclosed herein were obtained in one of two ways: (1) using an Inel XRG-3000 diffractometer equipped with a CPS (Curved Position Sensitive) detector with a 2θ range of 120°. Real time data were collected using Cu—Kα radiation. The tube voltage and amperage were set to 40 kV and 30 mA, respectively. The monochromator slit was set at 1-5 mm by 160 μm. The patterns are displayed from 2.5-40° 2θ. Samples were prepared for analysis by packing them into thin-walled glass capillaries. Each capillary was mounted onto a goniometer head that is motorized to permit spinning of the capillary during data acquisition. The sample analysis time is provided on the plots in the data section. Instrument calibration was performed using a silicon reference standard; or (2) using a PANalytical X'Pert Pro diffractometer. The specimen was analyzed using Cu radiation produced using an Optix long fine-focus source. An elliptically graded multilayer mirror was used to focus the Cu Kα x-rays of the source through the specimen and onto the detector. The specimen was sandwiched between 3-micron thick films, analyzed in transmission geometry, and rotated to optimize orientation statistics. A beam-stop was used to minimize the background generated by air scattering. Soller slits were used for the incident and diffracted beams to minimize axial divergence. Diffraction patterns were collected using a scanning position-sensitive detector (X'Celerator) located 240 mm from the specimen. The data-acquisition parameters of each diffraction pattern are displayed above the image of each pattern in the data section. Prior to the analysis a silicon specimen (NIST standard reference material 640c) was analyzed to verify the position of the silicon 111 peak.

As used herein, the term “DSC” refers to differential scanning calorimetry. DSC data disclosed herein were obtained using a TA Instruments differential scanning calorimeter Q2000. The sample was placed in an aluminum DSC pan, and the weight accurately recorded. The analysis parameters are listed on the plots in the data section. Indium metal was used as the calibration standard. Reported temperatures are at the transition maxima and are reported to the nearest degree.

As used herein, the term “¹H-NMR” refers to proton nuclear magnetic resonance spectroscopy. Solution proton nuclear magnetic resonance (¹H-NMR) spectra were collected from ˜5-50-mg samples dissolved in the appropriate deuterated solvent. The specific acquisition parameters are listed on the plot of the first full spectrum of each sample in the data section.

As used herein, the term “TGA” refers to thermogravimetric analysis. TGA data disclosed herein were obtained using a TA Instruments Q5000IR thermogravimetric analyzer. Each sample was placed in an aluminum sample pan and inserted into the TG furnace. The analysis parameters are listed on the plots in the data section. Nickel and Alumel™ were used as the calibration standards. Reported temperatures are at the transition maxima and are reported to the nearest degree. The transitions are reported to the nearest tenth of a percent.

As described herein, optical microscopy was performed using a Leica MZ12.5 stereomicroscope. Samples were viewed in situ or on a glass slide (covered in Paratone-N oil) through crossed polarizers and a first order red compensator using various objectives ranging from 0.8-10×.

As used herein, “Raman” refers to Raman spectroscopy. Raman spectra disclosed herein were acquired on a Raman accessory module interfaced to a Magna 960® Fourier transform infrared (FT-IR) spectrophotometer (Thermo Nicolet). These modules use an excitation wavelength of 1064 nm and an indium gallium arsenide (InGaAs) detector. The samples were prepared for analysis by placing the material in a glass tube and positioning the tube in a gold-coated tube holder in the accessory. A specified number of sample scans were collected using Happ-Genzel apodization. Specific parameters are printed on each spectrum in the data section. Wavelength calibration was performed using sulfur and cyclohexane. The specific parameters of each spectrum are provided on the attached figures.

As used herein, “IR” refers to infrared spectroscopy. IR spectra were acquired with a Magna-IR 860® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. Wavelength verification was performed using NIST SRM 1921b (polystyrene). An attenuated total reflectance (ATR) accessory (Thunderdome™, Thermo Spectra-Tech), with a germanium (Ge) crystal was used for data acquisition. The data acquisition parameters for each pattern are displayed above each spectrum. A background data set was acquired with a clean Ge crystal. A Log 1 /R (R=reflectance) spectrum was obtained by taking a ratio of these two data sets against each other.

Photo micrographs were obtained on a Leica DM 2500 P compound microscope equipped with a PAXcam 3 digital microscope camera controlled by PAX-it 7.1 software.

As used herein with respect to the various analytical techniques described herein and data generated therefrom, the term “substantially” the same as or similar to is meant to convey that a particular set of analytical data is, within acceptable scientific limits, is sufficiently similar to that disclosed herein such that one of skill in the art would appreciate that the crystalline salt form of the compound is the same as that of the present invention. One of skill in the art would appreciate that certain analytical techniques, such as, for example, XRPD, ¹H-NMR, DSC, TGA, IR, and Raman, will not produce exactly the same results every time due to, for example, instrumental variation, sample preparation, scientific error, etc. By way of example only, XRPD results (i.e. peak locations, intensities, and/or presence) may vary slightly from sample to sample, despite the fact that the samples are, within accepted scientific principles, the same form, and this may be due to, for example, preferred orientation or varying solvent or water content. It is well within the ability of those skilled in the art, looking at the data as a whole, to appreciate whether such differences indicate a different form, and thus determine whether analytical data being compared to those disclosed herein are substantially similar. In this regard, and as is commonly practiced within the scientific community, it is not intended that the exemplary analytical data of the novel salt forms of eperisone disclosed herein be met literally in order to determine whether comparative data represent the same form as those disclosed and claimed herein, such as, for example, whether each and every peak of an exemplary XRPD pattern of a novel crystalline salt of eperisone disclosed herein is present in the comparative data, in the same location, and/or of the same intensity. Rather, as discussed above, it is intended that those of skill in the art, using accepted scientific principles, will make a determination based on the data as a whole regarding whether comparative analytical data represent the same or a different form of any of the novel crystalline salts of eperisone disclosed herein.

It is to be understood that both the foregoing general description and the following detailed description are exemplary and explanatory only and are not restrictive of the invention, as claimed.

### BRIEF DESCRIPTION OF THE FIGURES

FIG. 1 is an XRPD pattern of crystalline racemic eperisone fumarate, according to one embodiment of the invention;

FIG. 2 is an XRPD pattern of crystalline racemic eperisone maleate, according to one embodiment of the invention;

FIG. 3 is an XRPD pattern of crystalline racemic eperisone mesylate, according to one embodiment of the invention;

FIG. 4 is an XRPD pattern of crystalline racemic eperisone succinate, according to one embodiment of the invention;

FIGS. 5A-5D are an ¹H-NMR spectrum of crystalline racemic eperisone fumarate, according to one embodiment of the invention;

FIGS. 6A-6E are an ¹H-NMR spectrum of crystalline racemic eperisone maleate, according to one embodiment of the invention;

FIGS. 7A-7F are an ¹H-NMR spectrum of crystalline racemic eperisone mesylate, according to one embodiment of the invention;

FIGS. 8A-8D are an ¹H-NMR spectrum of crystalline racemic eperisone succinate, according to one embodiment of the invention:

FIG. 9 is an FT-Raman spectrum of crystalline racemic eperisone fumarate, according to one embodiment of the invention;

FIG. 10 is an FT-Raman spectrum of crystalline racemic eperisone maleate, according to one embodiment of the invention;

FIG. 11 is an FT-Raman spectrum of crystalline racemic eperisone mesylate, according to one embodiment of the invention;

FIG. 12 is an FT-Raman spectrum of crystalline racemic eperisone succinate, according to one embodiment of the invention;

FIG. 13 is an IR spectrum of crystalline racemic eperisone fumarate, according to one embodiment of the invention;

FIG. 14 is an IR spectrum of crystalline racemic eperisone maleate, according to one embodiment of the invention;

FIG. 15 is an IR spectrum of crystalline racemic eperisone mesylate, according to one embodiment of the invention;

FIG. 16 is an IR spectrum of crystalline racemic eperisone succinate, according to one embodiment of the invention;

FIG. 17 is a DSC thermogram of crystalline racemic eperisone fumarate, according to one embodiment of the invention;

FIG. 18 is a DSC thermogram of crystalline racemic eperisone maleate, according to one embodiment of the invention;

FIG. 19 is a DSC thermogram of crystalline racemic eperisone mesylate, according to one embodiment of the invention;

FIG. 20 is a DSC thermogram of crystalline racemic eperisone succinate, according to one embodiment of the invention;

FIG. 21 is a TGA profile of crystalline racemic eperisone fumarate, according to one embodiment of the invention;

FIG. 22 is a TGA profile of crystalline racemic eperisone maleate, according to one embodiment of the invention;

FIG. 23 is a TGA profile of crystalline racemic eperisone mesylate, according to one embodiment of the invention;

FIG. 24 is a TGA profile of crystalline racemic eperisone succinate, according to one embodiment of the invention;

FIG. 25 is a photo micrograph of crystals of racemic eperisone fumarate, according to one embodiment of the invention;

FIG. 26 is a photo micrograph of crystals of racemic eperisone maleate, according to one embodiment of the invention;

FIG. 27 is a photo micrograph of crystals of racemic eperisone mesylate, according to one embodiment of the invention;

FIG. 28 is a photo micrograph of crystals of racemic eperisone succinate, according to one embodiment of the invention; and

FIG. 29 is a photo micrograph of crystals of racemic eperisone hydrochloride.

### DESCRIPTION OF EXEMPLARY EMBODIMENTS

The invention relates to novel crystalline salt forms of eperisone, including crystalline racemic eperisone fumarate, crystalline racemic eperisone maleate, crystalline racemic eperisone mesylate, and crystalline racemic eperisone succinate. Exemplary methods of preparation of the novel crystalline salt forms of eperisone according to various embodiments of the invention are described below in the examples.

In addition, pharmaceutical compositions containing the novel crystalline salt forms of eperisone, and their use in the treatment and/or prevention of various conditions including, for example, myotonic conditions, pain, and pathological muscle tension, as well as improving blood flow, are also disclosed.

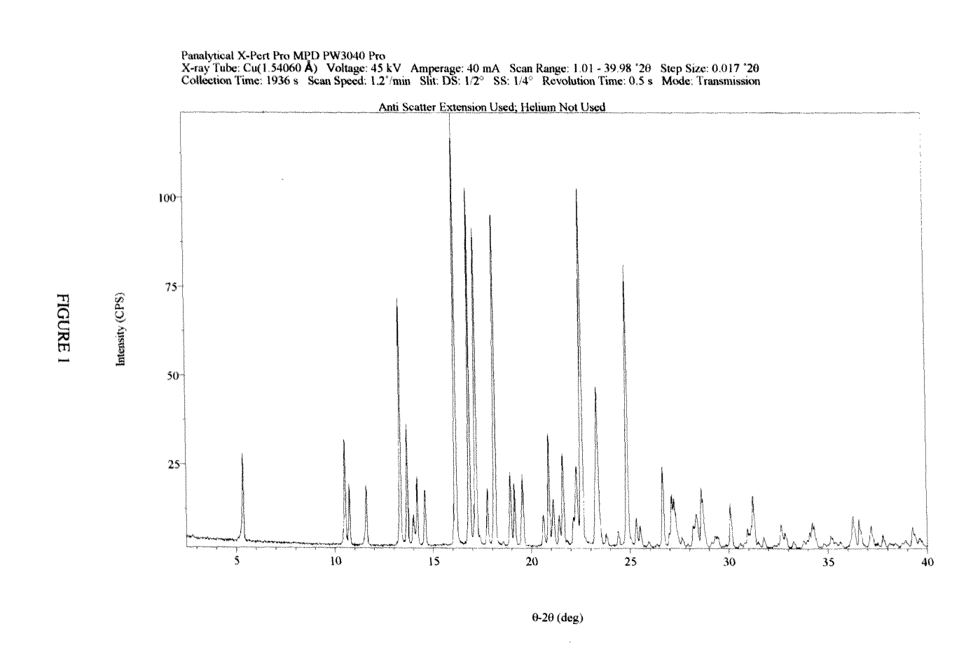

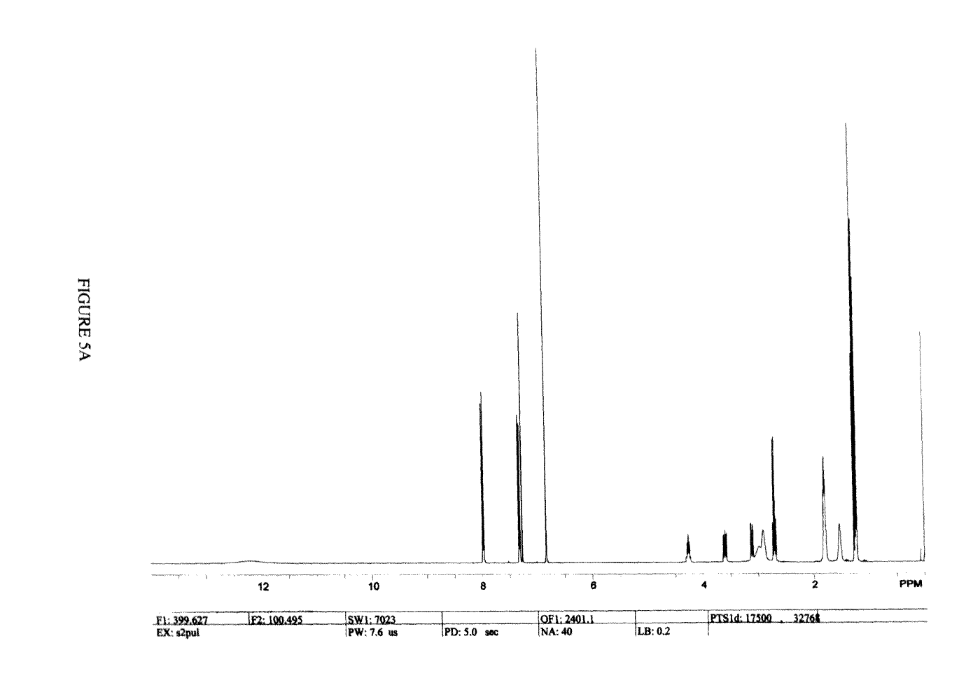

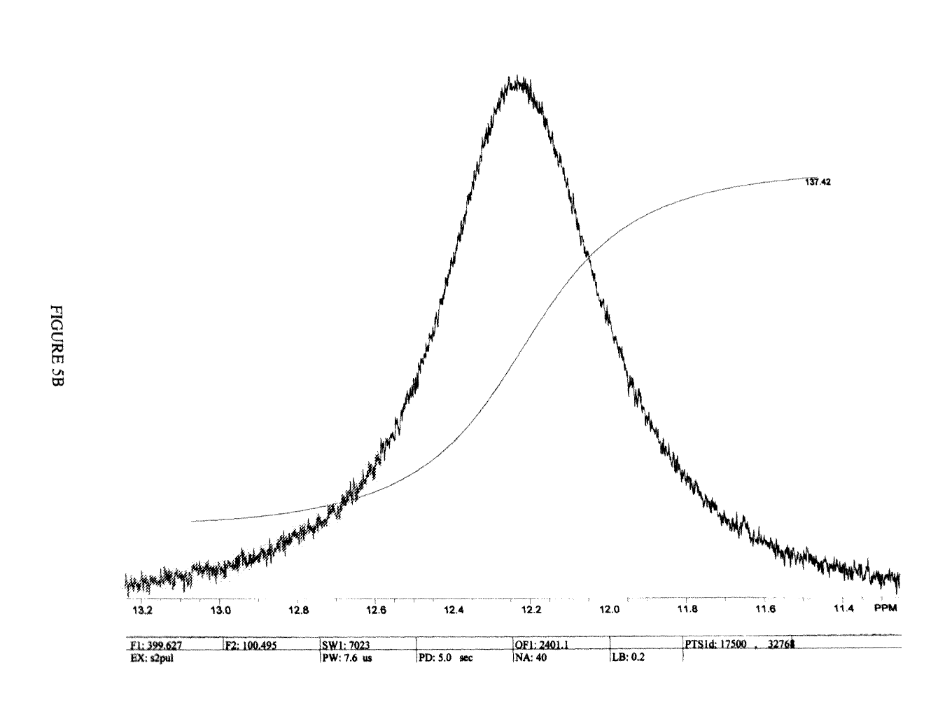

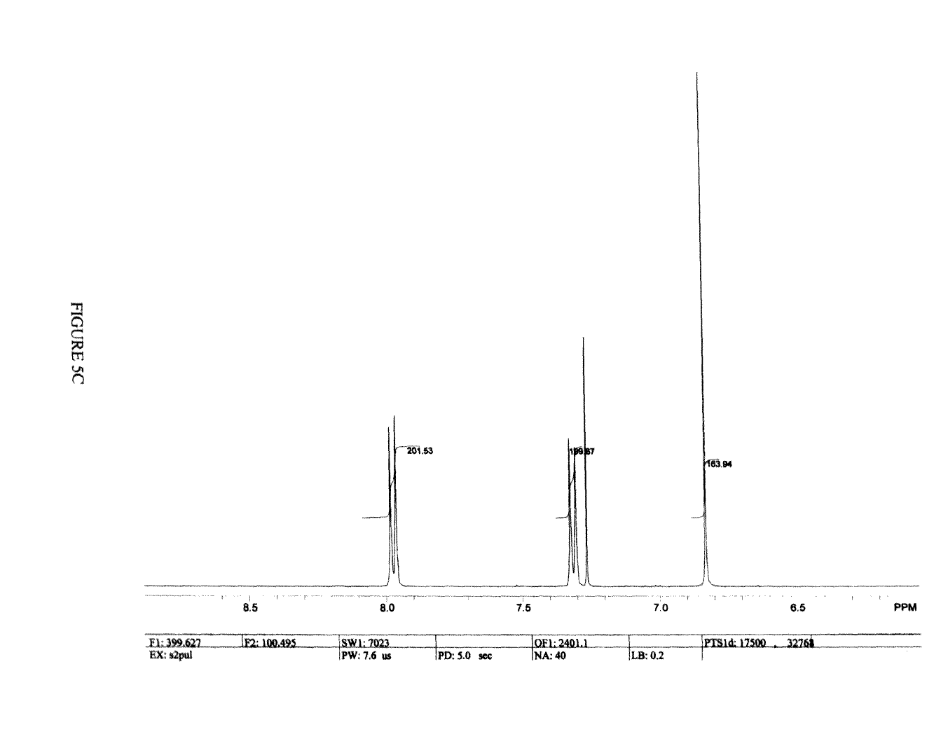

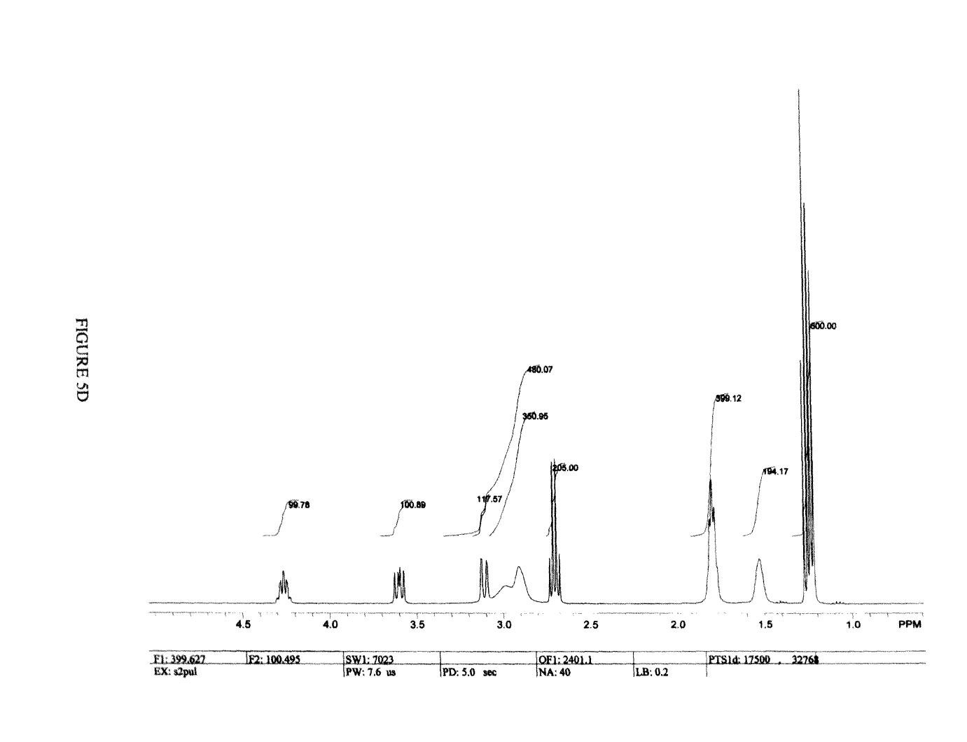

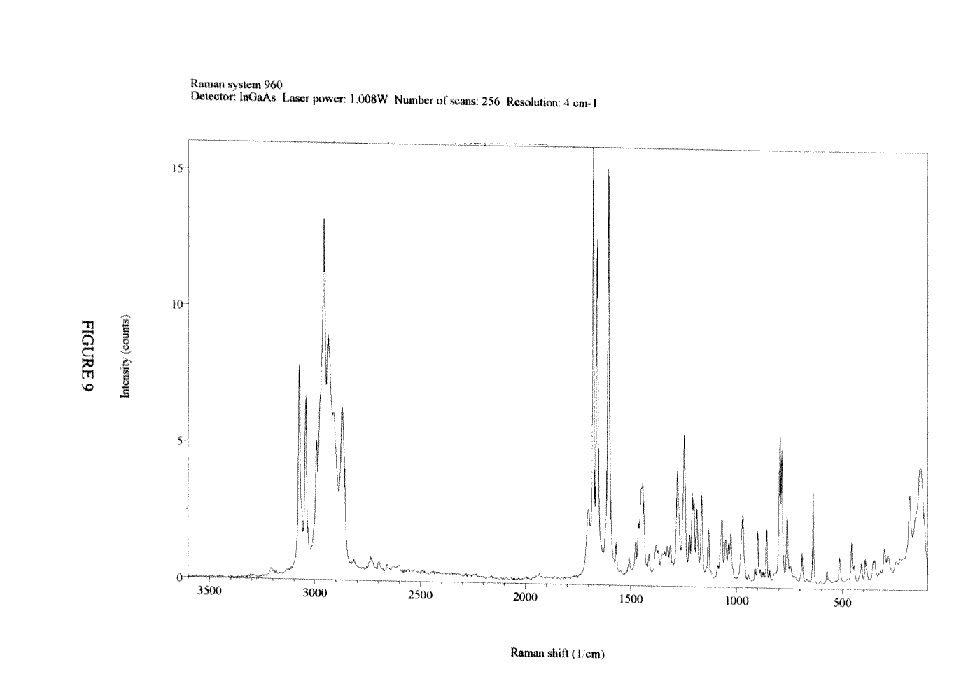

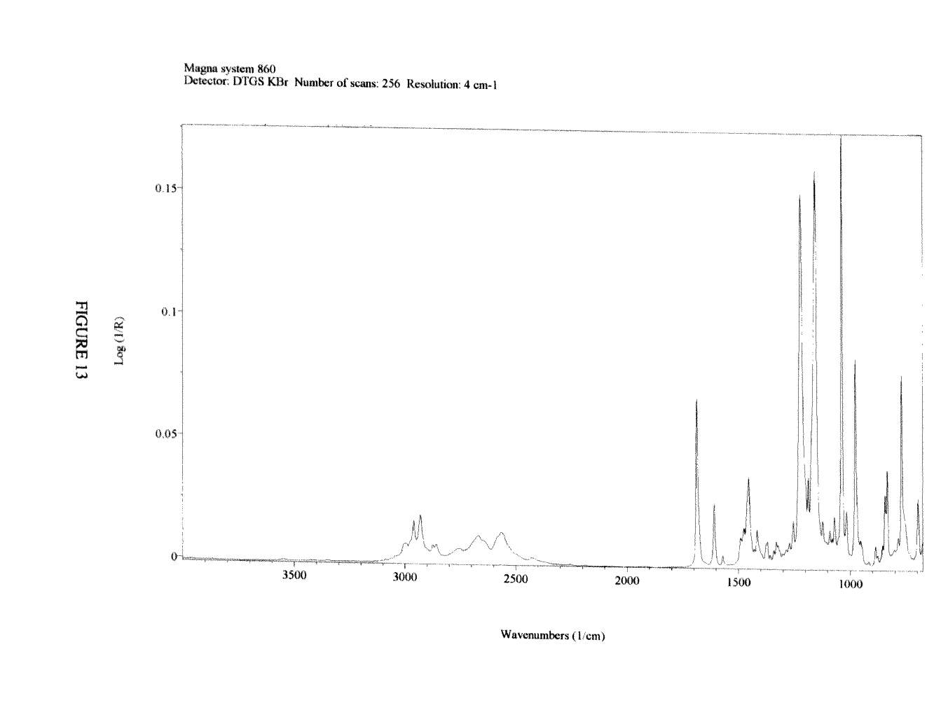

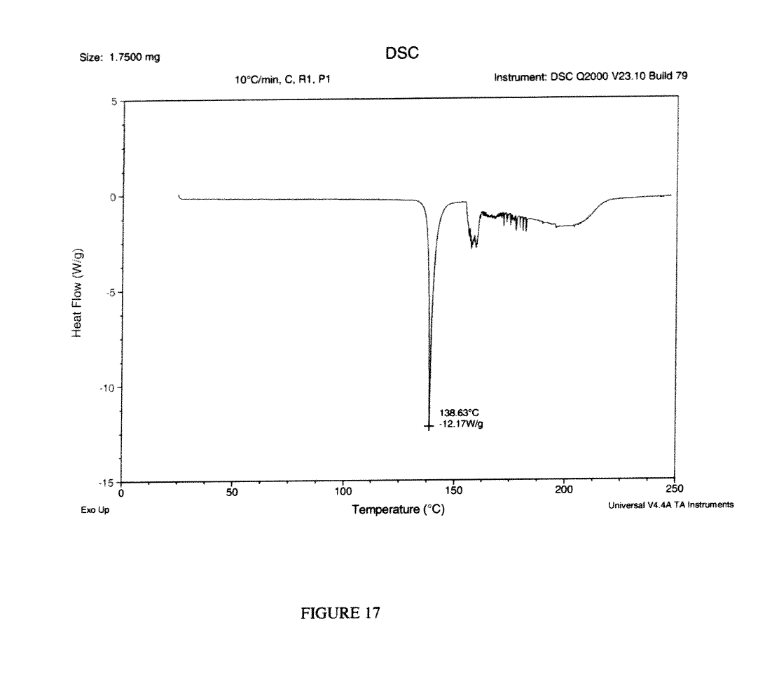

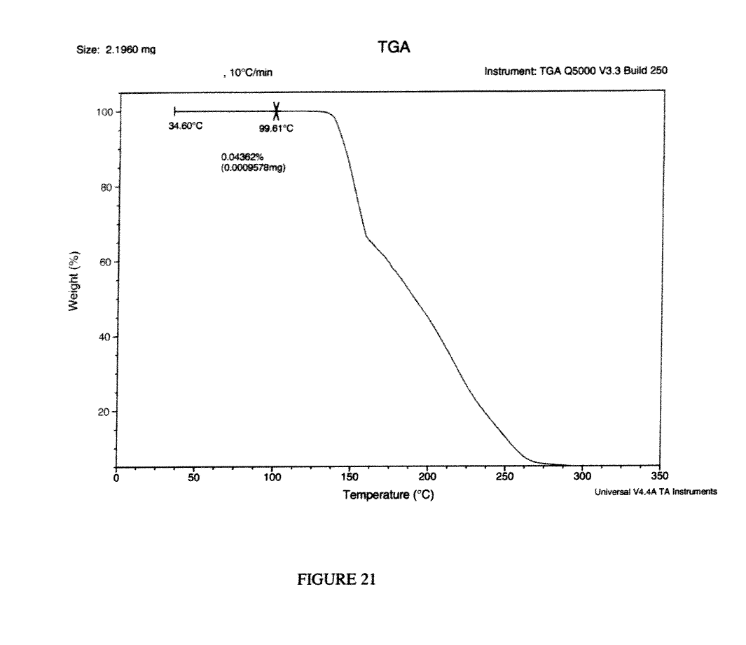

Crystalline racemic eperisone fumarate is characterized by an XRPD pattern substantially as shown in FIG. 1, an ¹H-NMR spectrum substantially as shown in FIGS. 5A-5D, a Raman spectra substantially as shown in FIG. 9, an IR spectrum substantially as shown in FIG. 13, a DSC thermogram substantially as shown in FIG. 17, and a TGA profile substantially as shown in FIG. 21. An exemplary listing of representative XRPD peaks of crystalline racemic eperisone fumarate according to an embodiment of the invention can be found in Table 1. An exemplary listing of representative NMR data, obtained in CDCl₃, can be found in Table 2.

TABLE 1

| Degrees 2θ | | d spacing Å | | Intensity % (I/Io) |
|---|---|---|---|---|
| 5.37 | ±0.2 | 16.457 | ±0.62 | 22 |
| 10.52 | ±0.2 | 8.412 | ±0.16 | 25 |
| 10.75 | ±0.2 | 8.229 | ±0.14 | 15 |
| 11.62 | ±0.2 | 7.616 | ±0.13 | 14 |
| 13.34 | ±0.2 | 6.637 | ±0.10 | 57 |
| 13.69 | ±0.2 | 6.467 | ±0.09 | 29 |
| 13.99 | ±0.2 | 6.329 | ±0.09 | 8 |
| 14.19 | ±0.2 | 6.240 | ±0.09 | 16 |
| 14.59 | ±0.2 | 6.070 | ±0.08 | 13 |
| 16.17 | ±0.2 | 5.483 | ±0.07 | 100 |
| 16.87 | ±0.2 | 5.256 | ±0.06 | 83 |
| 17.18 | ±0.2 | 5.160 | ±0.06 | 74 |
| 17.37 | ±0.2 | 5.106 | ±0.06 | 3 |
| 17.77 | ±0.2 | 4.992 | ±0.06 | 14 |
| 18.15 | ±0.2 | 4.887 | ±0.05 | 76 |
| 18.56 | ±0.2 | 4.782 | ±0.05 | 2 |
| 18.94 | ±0.2 | 4.686 | ±0.05 | 18 |
| 19.16 | ±0.2 | 4.633 | ±0.05 | 15 |
| 19.57 | ±0.2 | 4.535 | ±0.05 | 17 |
| 20.61 | ±0.2 | 4.309 | ±0.04 | 8 |
| 20.89 | ±0.2 | 4.252 | ±0.04 | 26 |
| 21.13 | ±0.2 | 4.205 | ±0.04 | 11 |
| 21.43 | ±0.2 | 4.147 | ±0.04 | 8 |
| 21.61 | ±0.2 | 4.112 | ±0.04 | 22 |
| 22.13 | ±0.2 | 4.017 | ±0.04 | 7 |
| 22.32 | ±0.2 | 3.984 | ±0.04 | 19 |
| 22.55 | ±0.2 | 3.943 | ±0.03 | 85 |
| 23.35 | ±0.2 | 3.810 | ±0.03 | 37 |
| 23.79 | ±0.2 | 3.741 | ±0.03 | 4 |
| 24.39 | ±0.2 | 3.650 | ±0.03 | 4 |
| 24.86 | ±0.2 | 3.582 | ±0.03 | 65 |
| 25.31 | ±0.2 | 3.519 | ±0.03 | 7 |
| 25.51 | ±0.2 | 3.492 | ±0.03 | 5 |
| 25.94 | ±0.2 | 3.435 | ±0.03 | 1 |
| 26.34 | ±0.2 | 3.383 | ±0.03 | 1 |
| 26.66 | ±0.2 | 3.344 | ±0.02 | 19 |
| 27.11 | ±0.2 | 3.289 | ±0.02 | 12 |
| 27.23 | ±0.2 | 3.275 | ±0.02 | 12 |
| 27.63 | ±0.2 | 3.229 | ±0.02 | 3 |
| 27.91 | ±0.2 | 3.196 | ±0.02 | 1 |
| 28.20 | ±0.2 | 3.165 | ±0.02 | 5 |
| 28.35 | ±0.2 | 3.148 | ±0.02 | 8 |
| 28.63 | ±0.2 | 3.118 | ±0.02 | 14 |

TABLE 2

| | peak | | coupling | number |
|---|---|---|---|---|
| | position | | constant | of |
| Protons | (ppm) | mutiplicity | (Hz) | protons |
| 2 × CH₃ | 1.22-1.27 | multiplet | — | 6 |
| | | (overlapping | | |
| | | triplets) | | |
| 3 × CH₂ | 1.53 | broad singlet | — | 2 |
| | 1.77-1.81 | multiplet | — | 4 |
| CH₂CH₃ | 2.70 | quartet | 8 | 2 |
| 3 × CH₂N | ~2.8-3.1 | broad multiplet | — | 4 |
| | 3.10-3.13 | multiplet | — | 1 |
| | 3.57-3.63 | multiplet | — | 1 |
| CH | 4.23-4.31 | multiplet | — | 1 |
| fumarate CH | 6.83 | singlet | — | ~1.6 |
| aromatic | 7.31 | doublet | 8 | 2 |
| aromatic | 7.97 | doublet | 8 | 2 |
| exchangeable | 12.2 | broad singlet | — | — |
| protons | | | | |

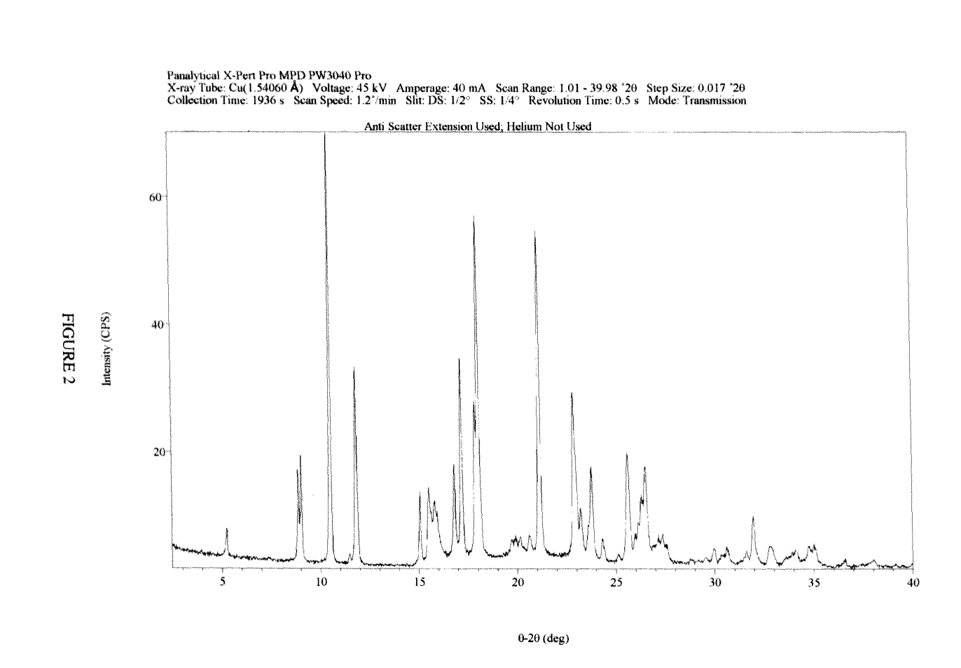

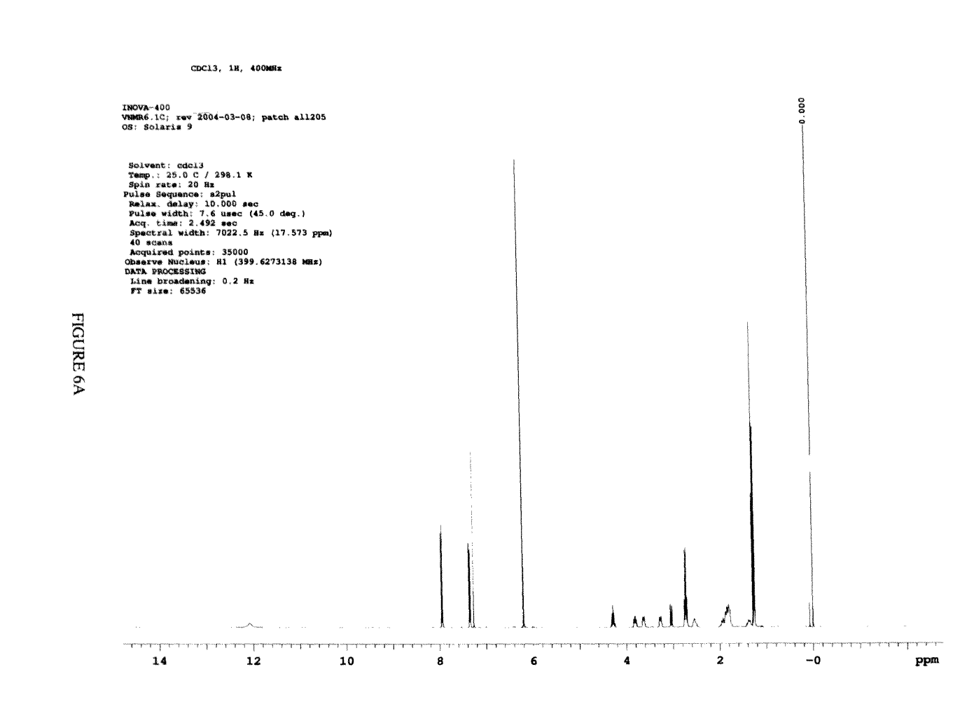

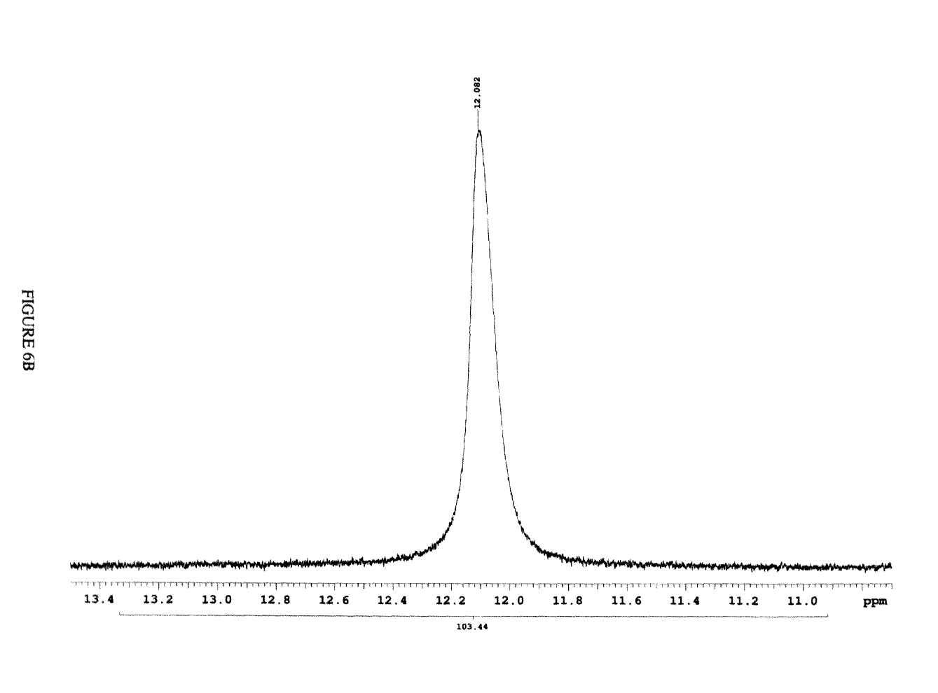

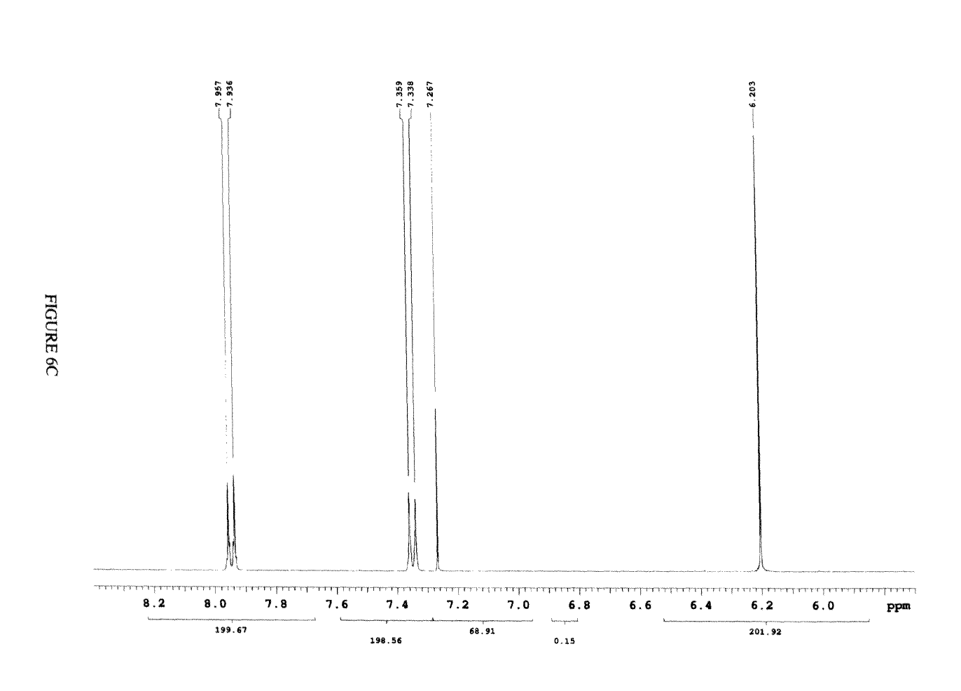

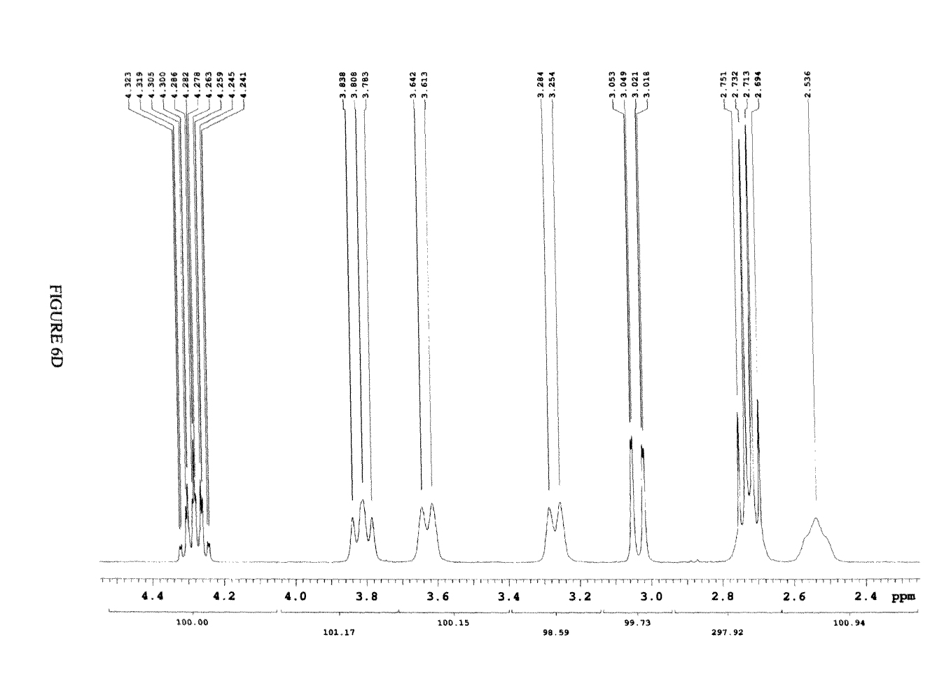

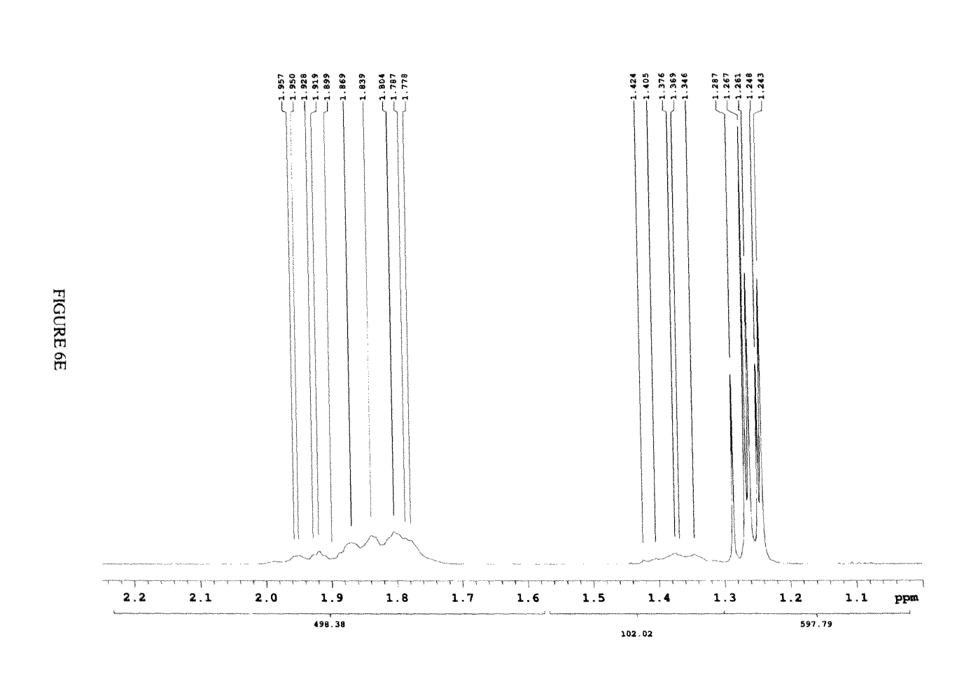

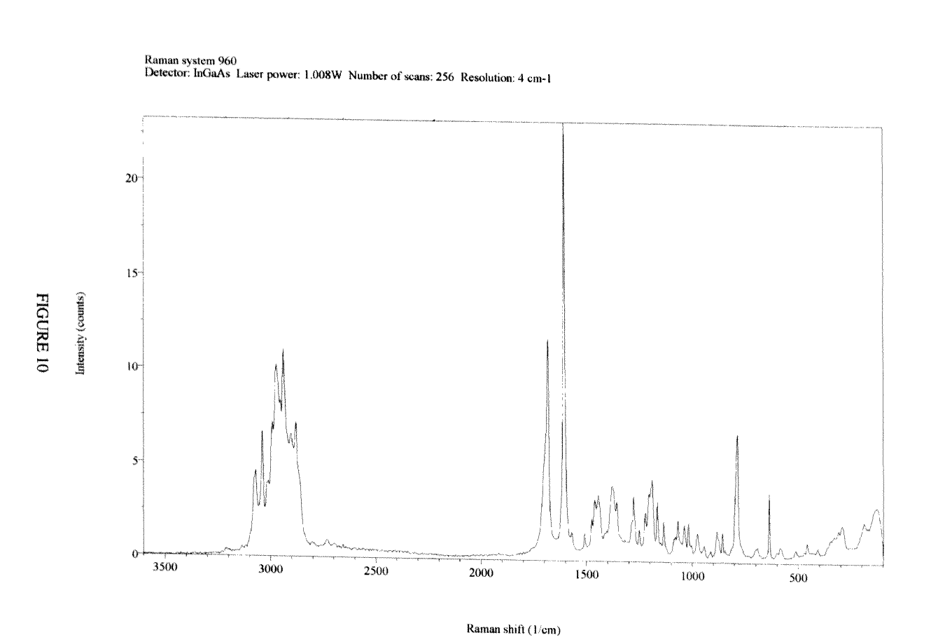

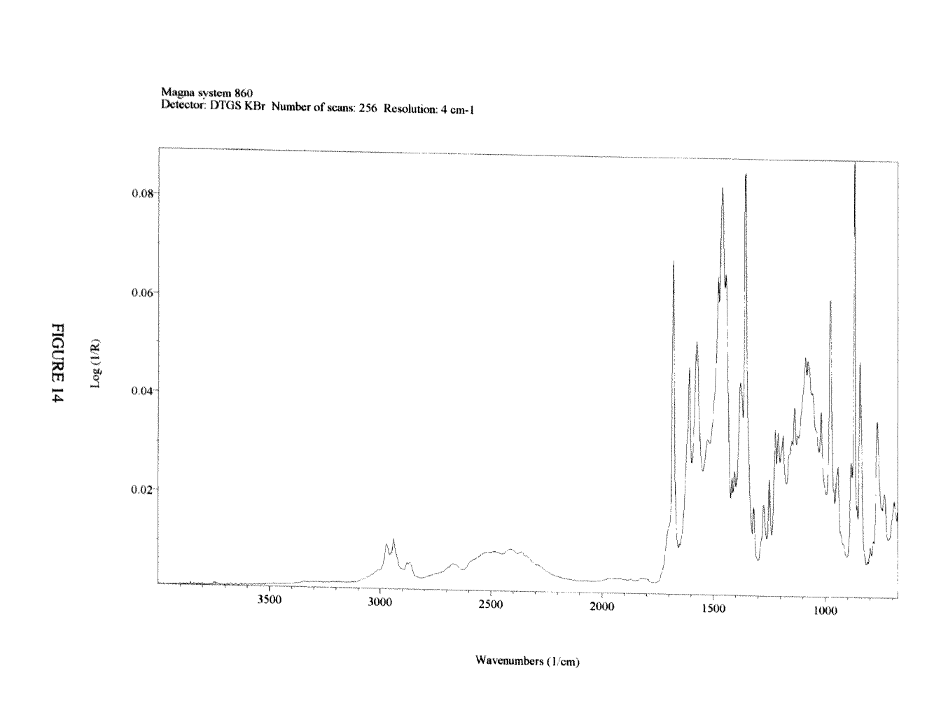

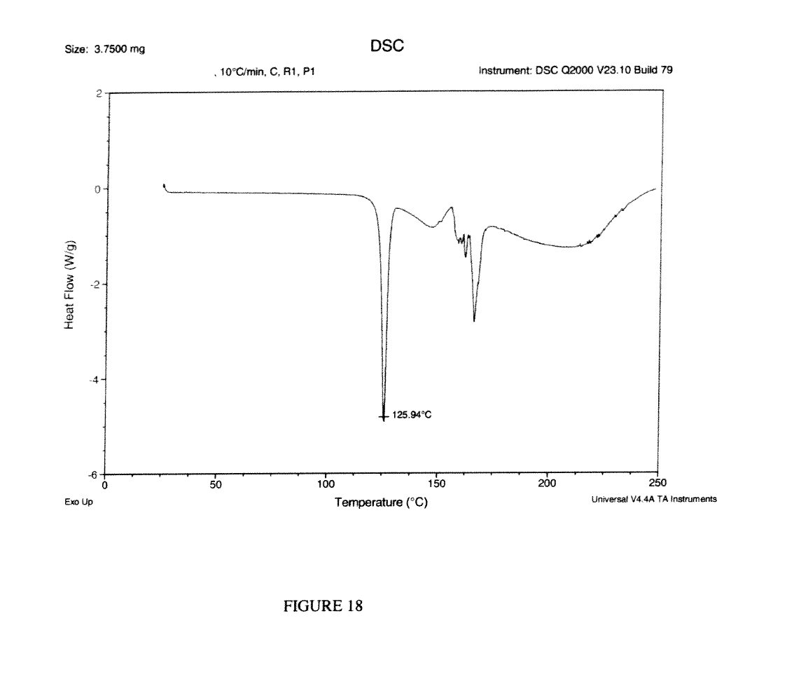

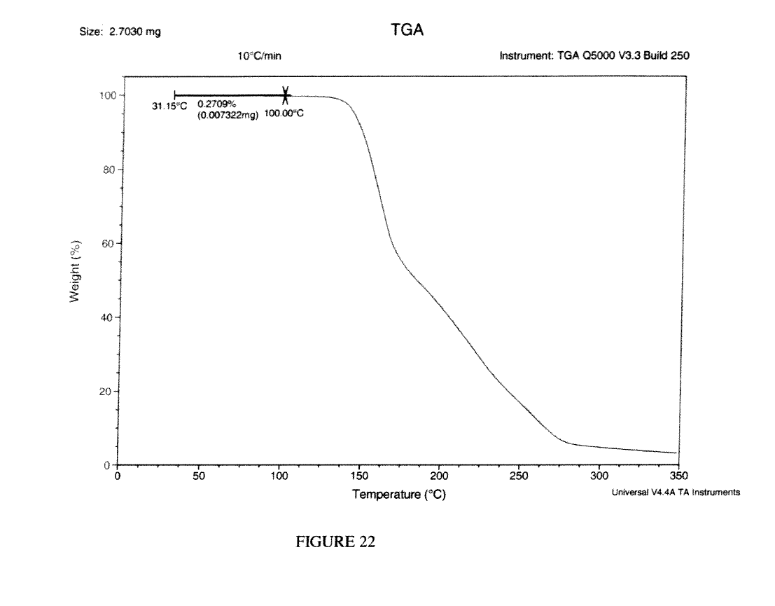

Crystalline racemic eperisone maleate is characterized by an XRPD pattern substantially as shown in FIG. 2, an ¹H-NMR spectrum substantially as shown in FIGS. 6A-6E, a Raman spectra substantially as shown in FIG. 10, an IR spectrum substantially as shown in FIG. 14, a DSC thermogram substantially as shown in FIG. 18, and a TGA profile substantially as shown in FIG. 22. An exemplary listing of representative XRPD peaks of crystalline racemic eperisone maleate according to an embodiment of the invention can be found in Table 3. An exemplary listing of representative NMR data, obtained in CDCl₃, can be found in Table 4.

TABLE 3

| Degrees 2θ | | d spacing Å | | Intensity % (I/Io) |
|---|---|---|---|---|
| 5.25 | ±0.2 | 16.823 | ±0.65 | 9 |
| 8.90 | ±0.2 | 9.941 | ±0.23 | 23 |
| 9.03 | ±0.2 | 9.794 | ±0.22 | 26 |
| 10.53 | ±0.2 | 8.398 | ±0.16 | 100 |
| 11.47 | ±0.2 | 7.715 | ±0.14 | 3 |
| 11.84 | ±0.2 | 7.476 | ±0.13 | 46 |
| 15.08 | ±0.2 | 5.876 | ±0.08 | 18 |
| 15.53 | ±0.2 | 5.706 | ±0.07 | 19 |
| 15.83 | ±0.2 | 5.598 | ±0.07 | 16 |
| 16.83 | ±0.2 | 5.267 | ±0.06 | 24 |
| 17.20 | ±0.2 | 5.155 | ±0.06 | 48 |
| 17.89 | ±0.2 | 4.959 | ±0.06 | 38 |
| 18.04 | ±0.2 | 4.918 | ±0.05 | 81 |
| 19.72 | ±0.2 | 4.501 | ±0.05 | 7 |
| 19.91 | ±0.2 | 4.460 | ±0.04 | 8 |
| 20.18 | ±0.2 | 4.401 | ±0.04 | 7 |
| 20.61 | ±0.2 | 4.309 | ±0.04 | 8 |
| 21.15 | ±0.2 | 4.202 | ±0.04 | 77 |
| 22.88 | ±0.2 | 3.886 | ±0.03 | 40 |
| 23.22 | ±0.2 | 3.831 | ±0.03 | 14 |
| 23.79 | ±0.2 | 3.741 | ±0.03 | 24 |
| 24.32 | ±0.2 | 3.660 | ±0.03 | 7 |
| 25.12 | ±0.2 | 3.545 | ±0.03 | 3 |
| 25.62 | ±0.2 | 3.477 | ±0.03 | 26 |
| 26.33 | ±0.2 | 3.385 | ±0.03 | 17 |
| 26.54 | ±0.2 | 3.358 | ±0.02 | 24 |
| 27.16 | ±0.2 | 3.283 | ±0.02 | 8 |
| 27.38 | ±0.2 | 3.258 | ±0.02 | 8 |

TABLE 4

| | peak | | coupling | number |
|---|---|---|---|---|
| | position | | constant | of |
| Protons | (ppm) | multiplicity | (Hz) | protons |
| 2 × CH₃ | 1.24-1.29 | multiplet | — | 6 |
| | | (overlapping | | |
| | | triplets) | | |
| 3 × CH₂ | 1.35-1.42 | multiplet | — | 1 |
| | 1.78-1.96 | multiplet | — | 5 |
| 0.5 × CH₂N | 2.54 | broad multiplet | — | 1 |
| CH₂CH₃ and | 2.72 | quartet on top of | 8 | 3 |
| 0.5 × CH₂N | | broad multiplet | | |
| 2 × CH₂N | 3.02-3.05 | multiplet | — | 1 |
| | 3.25-3.28 | multiplet | — | 1 |
| | 3.61-3.64 | multiplet | — | 1 |
| | 3.78-3.84 | multiplet | — | 1 |
| CH | 4.24-4.32 | multiplet | — | 1 |
| maleate CH | 6.20 | singlet | — | 2 |
| aromatic | 7.35 | doublet | 8 | 2 |
| aromatic | 7.95 | doublet | 8 | 2 |
| exchangeable | 12.08 | broad singlet | — | — |
| protons | | | | |

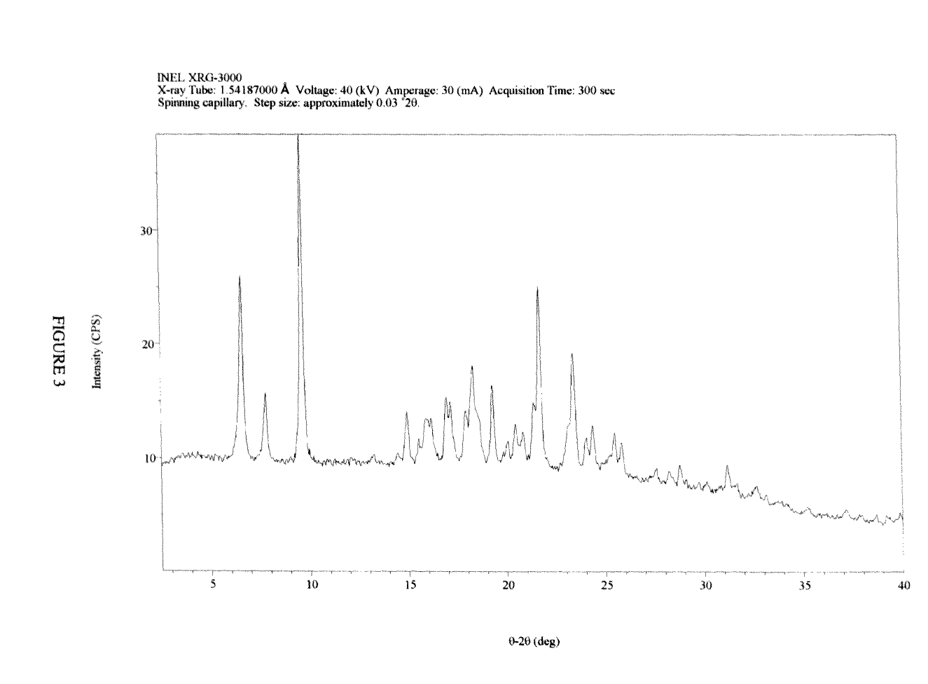

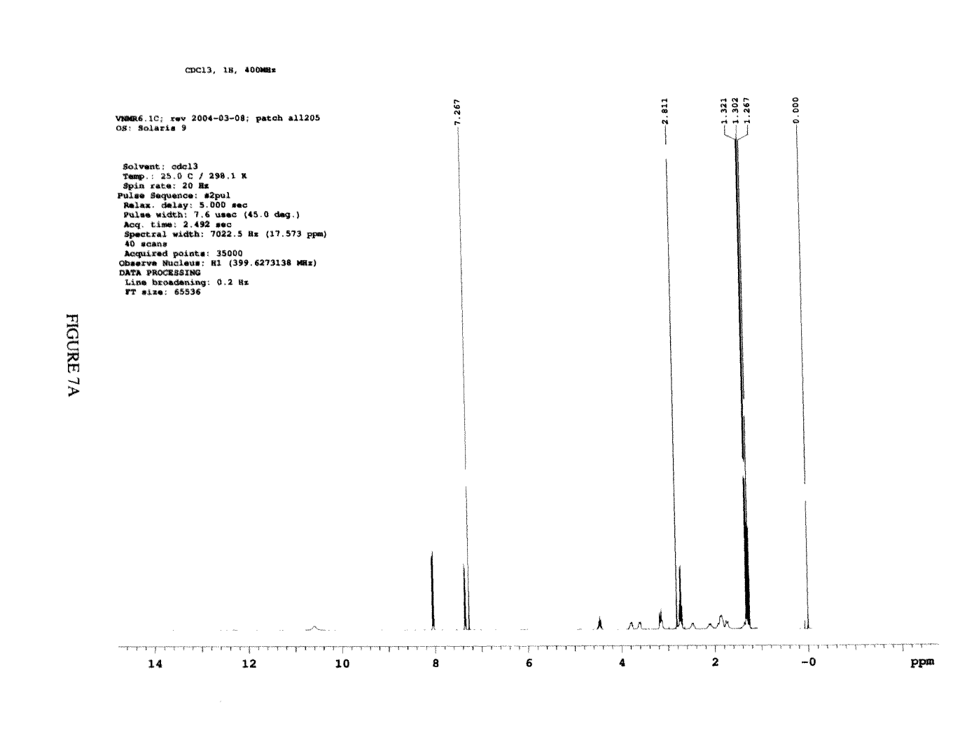

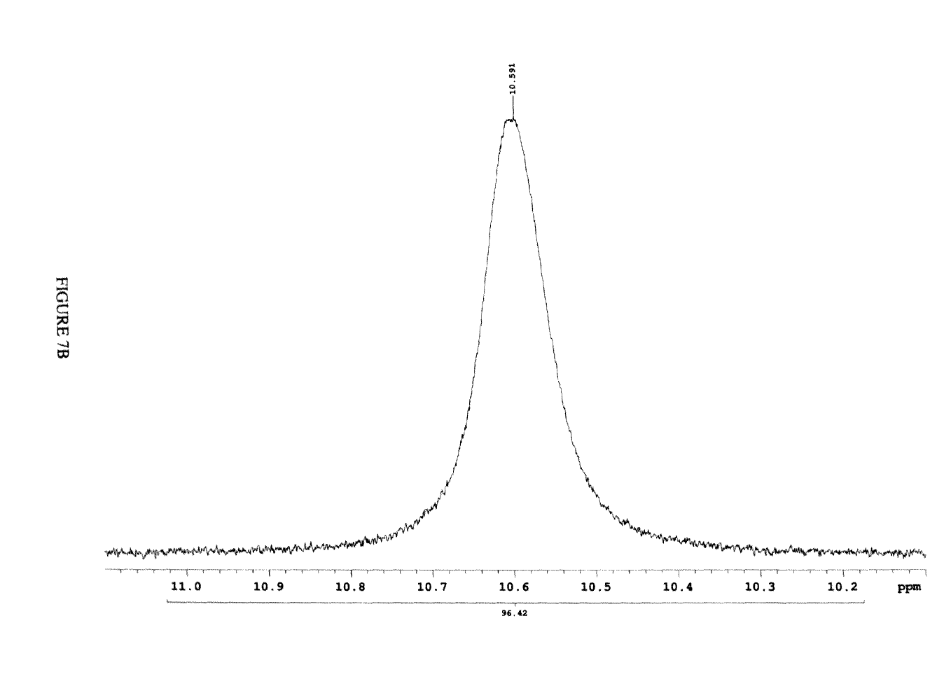

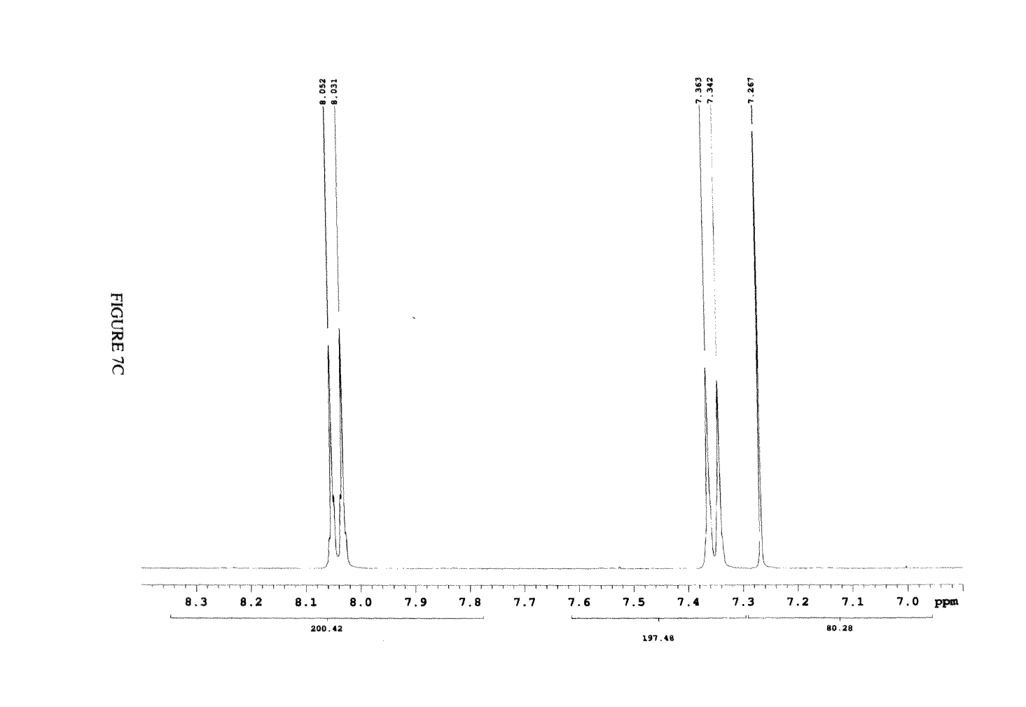

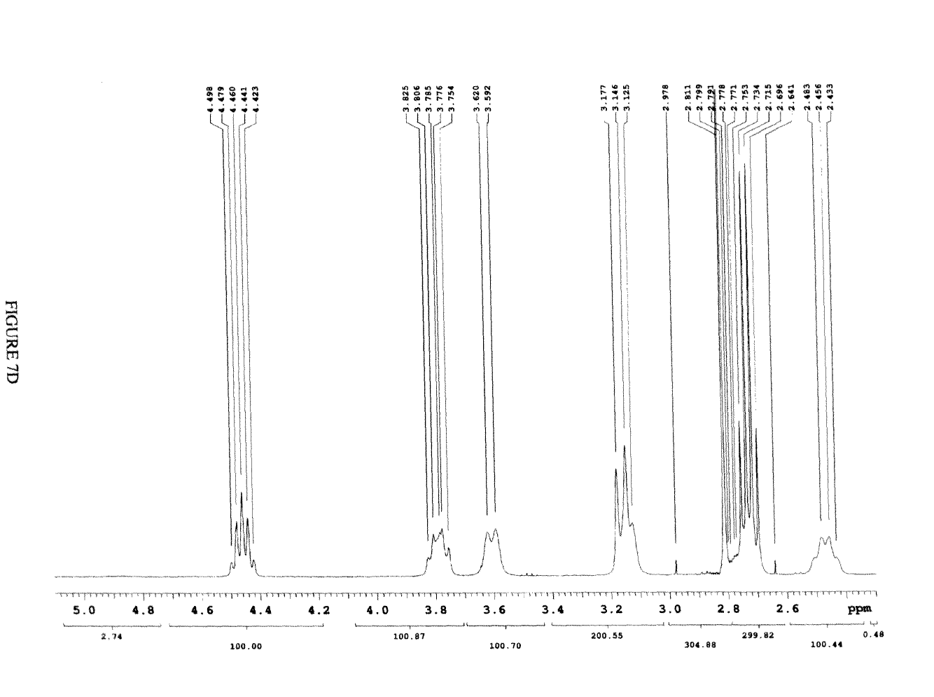

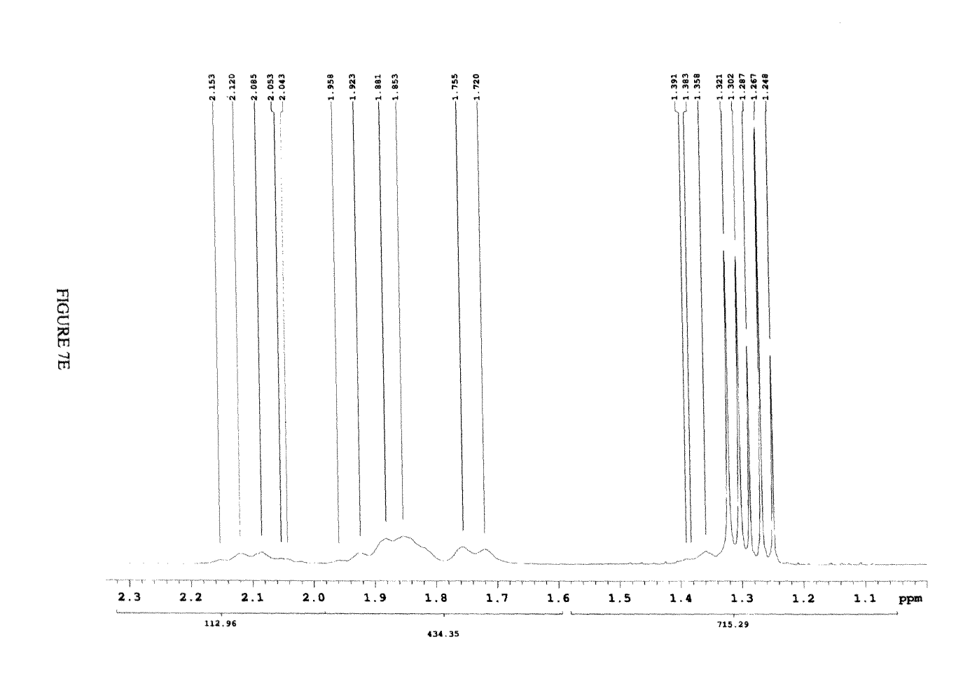

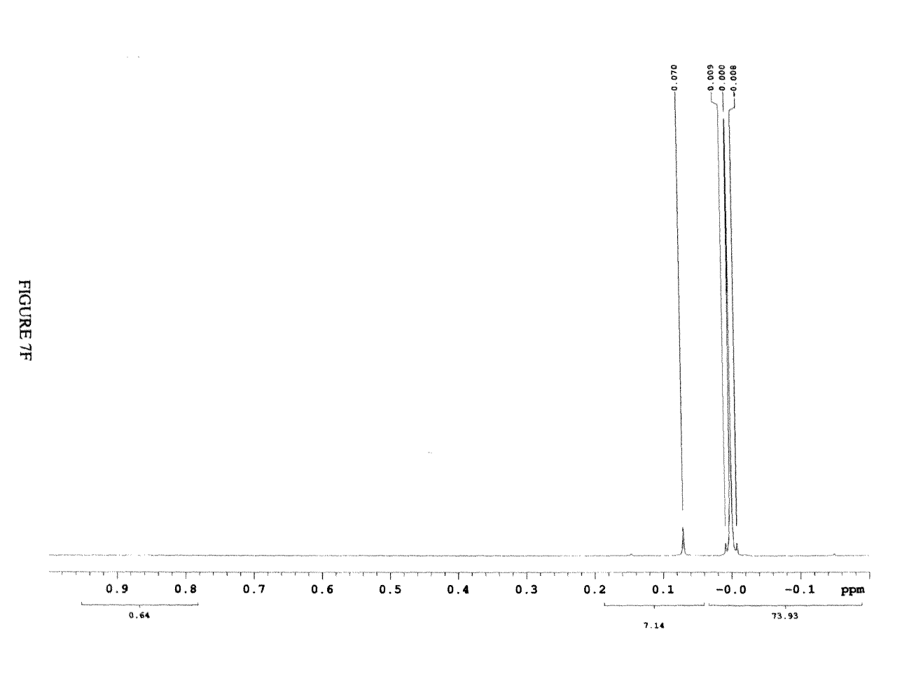

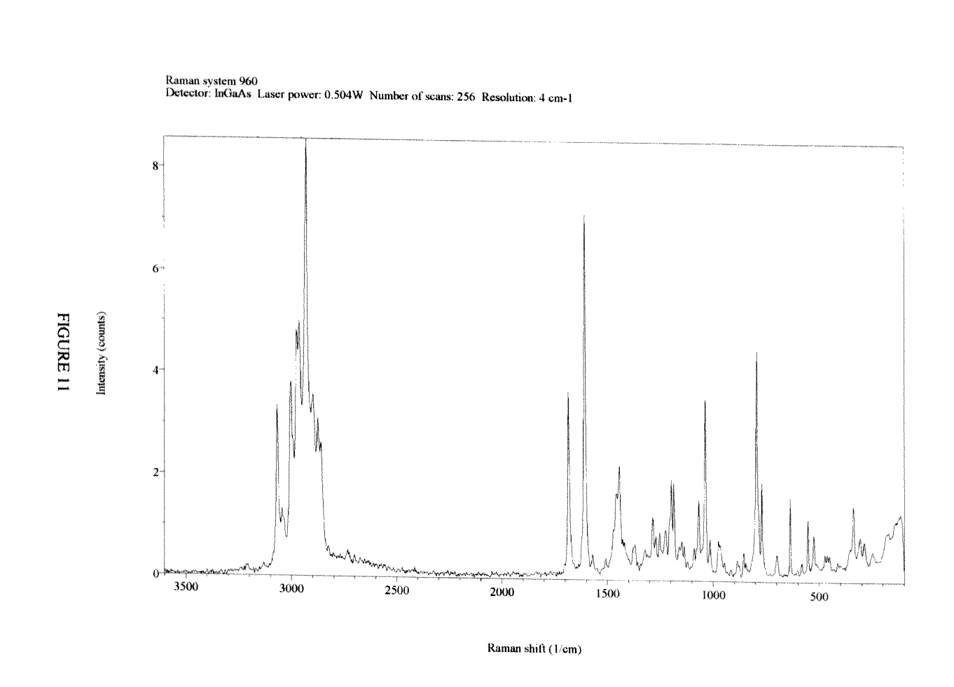

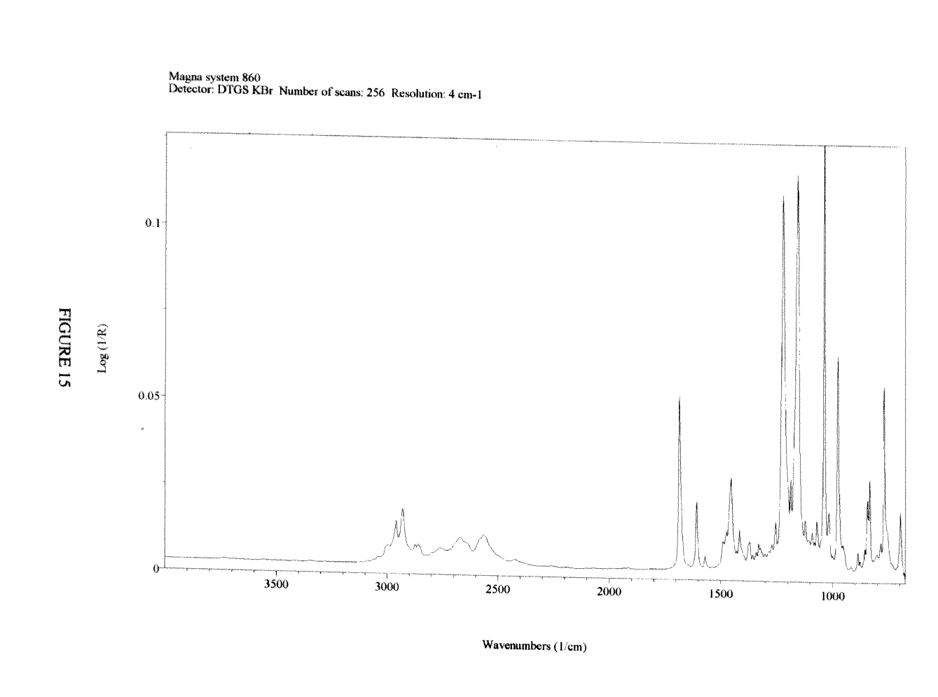

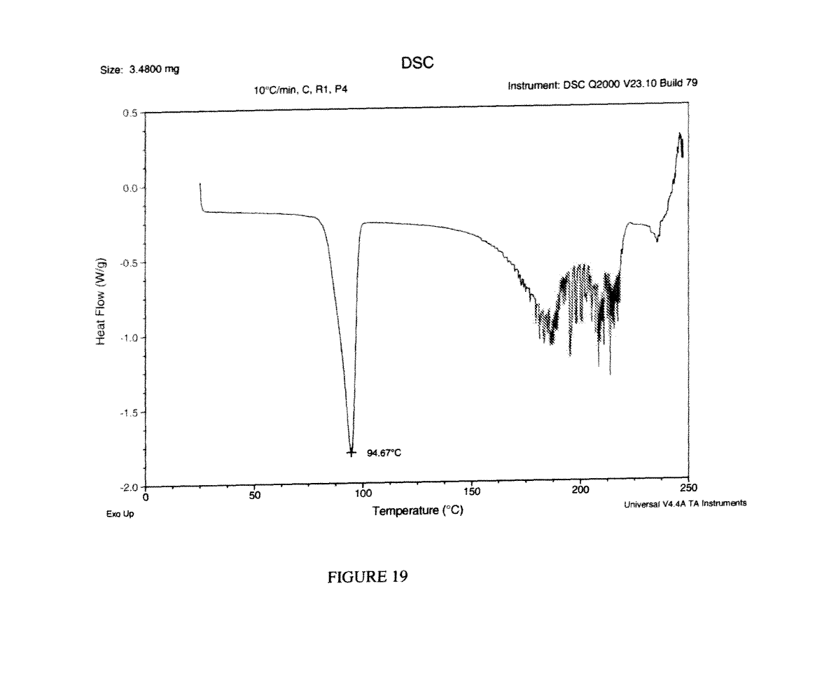

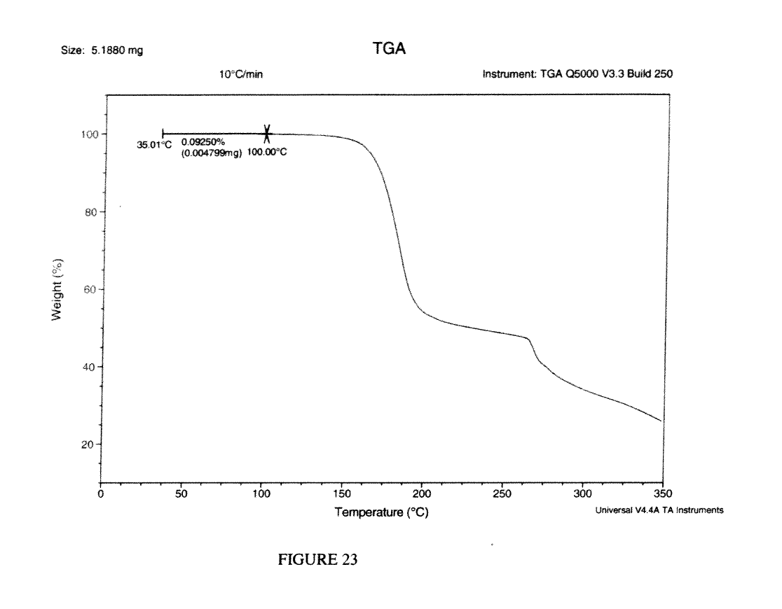

Crystalline racemic eperisone mesylate is characterized by an XRPD pattern substantially as shown in FIG. 3, an ¹H-NMR spectrum substantially as shown in FIGS. 7A-7F, a Raman spectra substantially as shown in FIG. 11, an IR spectrum substantially as shown in FIG. 15, a DSC thermogram substantially as shown in FIG. 19, and a TGA profile substantially as shown in FIG. 23. An exemplary listing of representative D peaks of crystalline racemic eperisone mesylate according to an embodiment of the invention can be found in Table 5. An exemplary listing of representative NMR data, obtained in CDCl₃, can be found in Table 6.

TABLE 5

| Degrees 2θ | | d spacing Å | | Intensity % (I/Io) |
|---|---|---|---|---|
| 6.60 | ±0.2 | 13.393 | ±0.41 | 57 |
| 7.80 | ±0.2 | 11.335 | ±0.29 | 21 |
| 9.69 | ±0.2 | 9.128 | ±0.19 | 100 |
| 13.26 | ±0.2 | 6.677 | ±0.10 | 3 |
| 14.46 | ±0.2 | 6.126 | ±0.08 | 3 |
| 14.97 | ±0.2 | 5.918 | ±0.08 | 16 |
| 15.57 | ±0.2 | 5.691 | ±0.07 | 8 |
| 15.93 | ±0.2 | 5.564 | ±0.07 | 14 |
| 16.20 | ±0.2 | 5.471 | ±0.07 | 14 |
| 16.98 | ±0.2 | 5.222 | ±0.06 | 20 |
| 17.19 | ±0.2 | 5.159 | ±0.06 | 19 |
| 17.94 | ±0.2 | 4.945 | ±0.05 | 16 |
| 18.33 | ±0.2 | 4.840 | ±0.05 | 30 |
| 18.63 | ±0.2 | 4.763 | ±0.05 | 14 |
| 19.32 | ±0.2 | 4.594 | ±0.05 | 24 |
| 19.86 | ±0.2 | 4.471 | ±0.04 | 4 |
| 20.10 | ±0.2 | 4.418 | ±0.04 | 7 |
| 20.49 | ±0.2 | 4.335 | ±0.04 | 13 |
| 20.88 | ±0.2 | 4.254 | ±0.04 | 10 |
| 21.42 | ±0.2 | 4.148 | ±0.04 | 20 |
| 21.72 | ±0.2 | 4.092 | ±0.04 | 55 |
| 23.19 | ±0.2 | 3.836 | ±0.03 | 14 |
| 23.43 | ±0.2 | 3.797 | ±0.03 | 36 |
| 24.06 | ±0.2 | 3.699 | ±0.03 | 11 |
| 24.39 | ±0.2 | 3.650 | ±0.03 | 15 |
| 25.26 | ±0.2 | 3.526 | ±0.03 | 7 |
| 25.50 | ±0.2 | 3.493 | ±0.03 | 14 |
| 25.86 | ±0.2 | 3.445 | ±0.03 | 11 |
| 27.60 | ±0.2 | 3.232 | ±0.02 | 5 |
| 28.23 | ±0.2 | 3.161 | ±0.02 | 5 |
| 28.77 | ±0.2 | 3.103 | ±0.02 | 7 |

TABLE 6

| | peak | | coupling | number |
|---|---|---|---|---|
| | position | | constant | of |
| Protons | (ppm) | multiplicity | (Hz) | protons |
| 2 × CH₃ and | 1.25-1.39 | overlapping | — | 7 |
| 0.5 × CH₂ | | triplets on top of | | |
| | | multiplet | | |
| 2.5 × CH₂ | 1.72-1.76 | multiplet | — | 4 |
| | 1.85-1.96 | multiplet | — | |
| | 2.04-2.15 | multiplet | — | 1 |
| 0.5 × CH₂N | 2.43-2.48 | multiplet | — | 1 |
| CH₂CH₃ and | 2.70-2.75 | quartet | 8 | 3 |
| 0.5 × CH₂N | | on top of broad | | |
| | | multiplet | | |
| CH₃SO₃ | 2.81 | singlet | — | 2 |
| 2 × CH₂N | 3.13-3.18 | multiplet | — | 2 |
| | 3.59-3.62 | multiplet | — | 1 |
| | 3.75-3.83 | multiplet | — | 1 |
| CH | 4.42-4.50 | multiplet | — | 1 |
| aromatic | 7.35 | doublet | 8 | 2 |
| aromatic | 8.04 | doublet | 8 | 2 |
| exchangeable | 10.59 | broad singlet | — | — |
| protons | | | | |

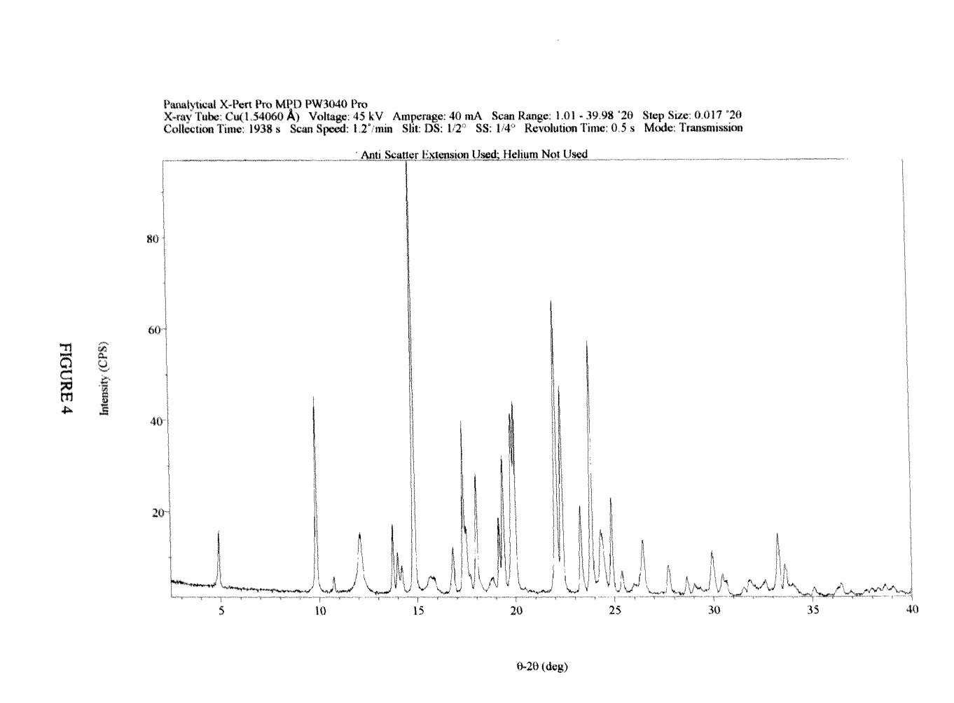

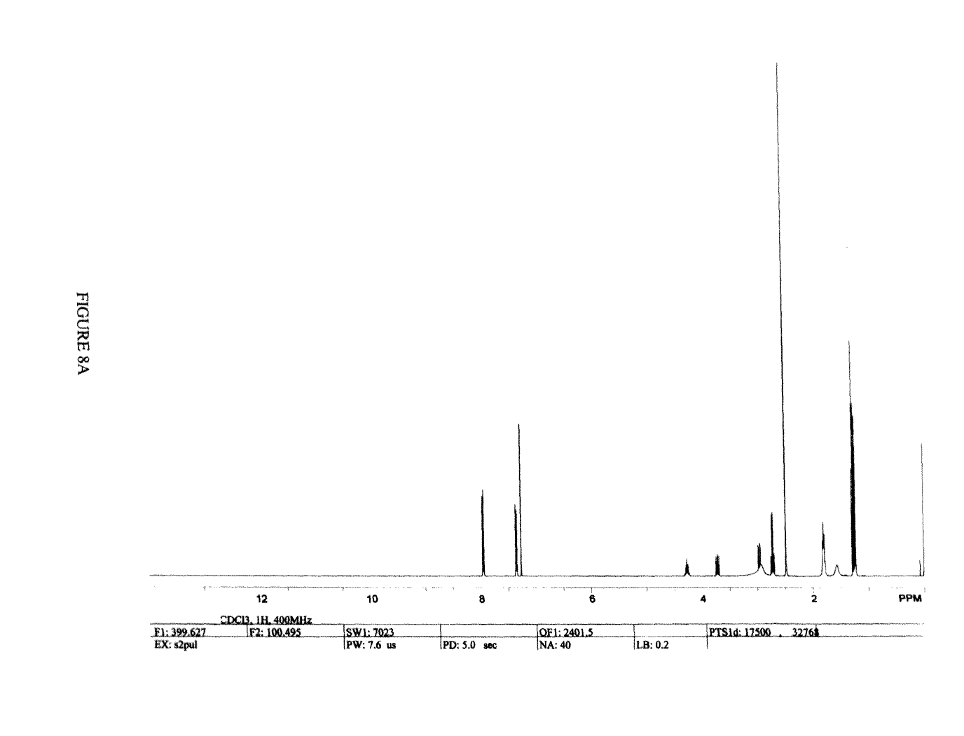

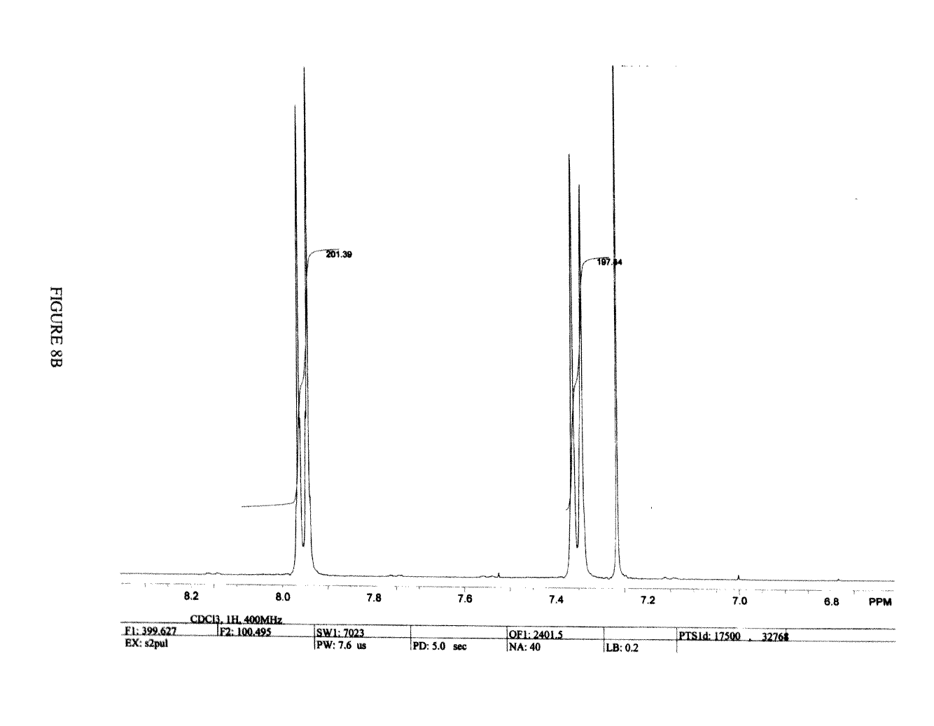

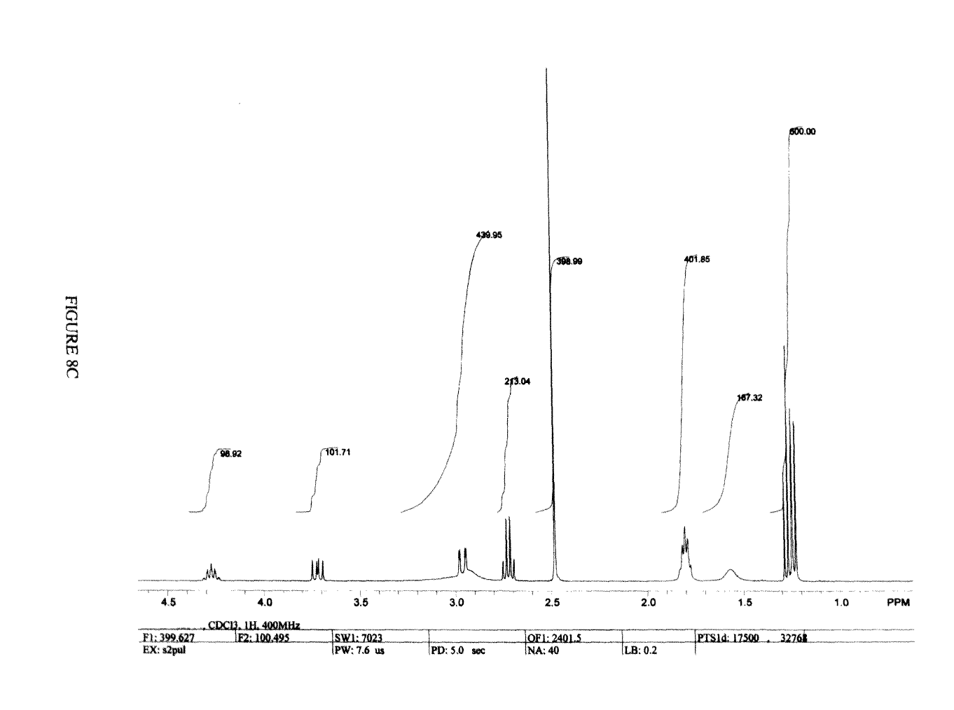

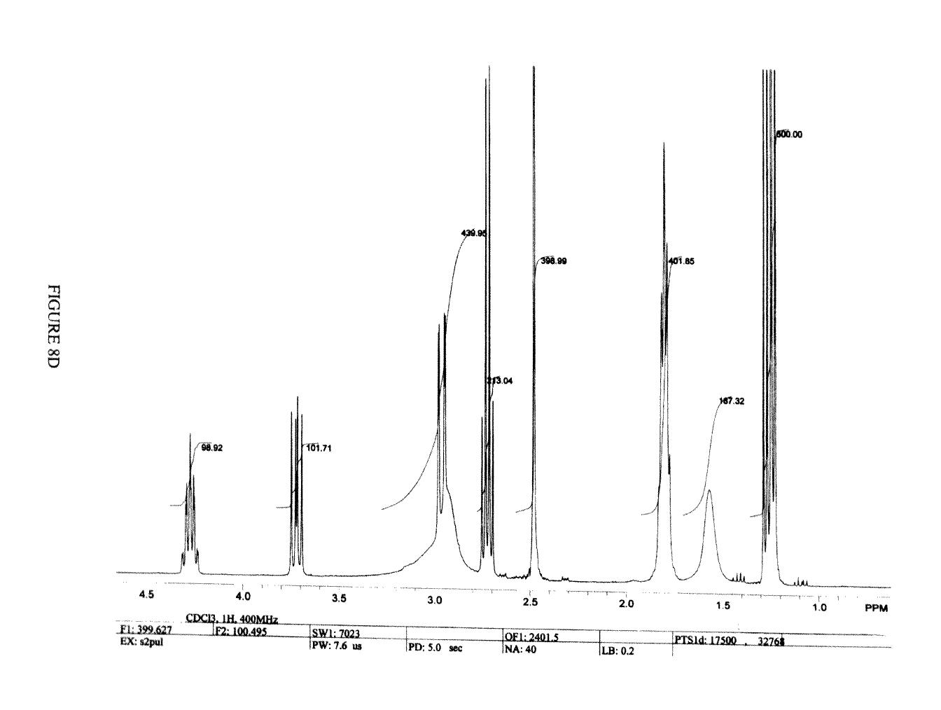

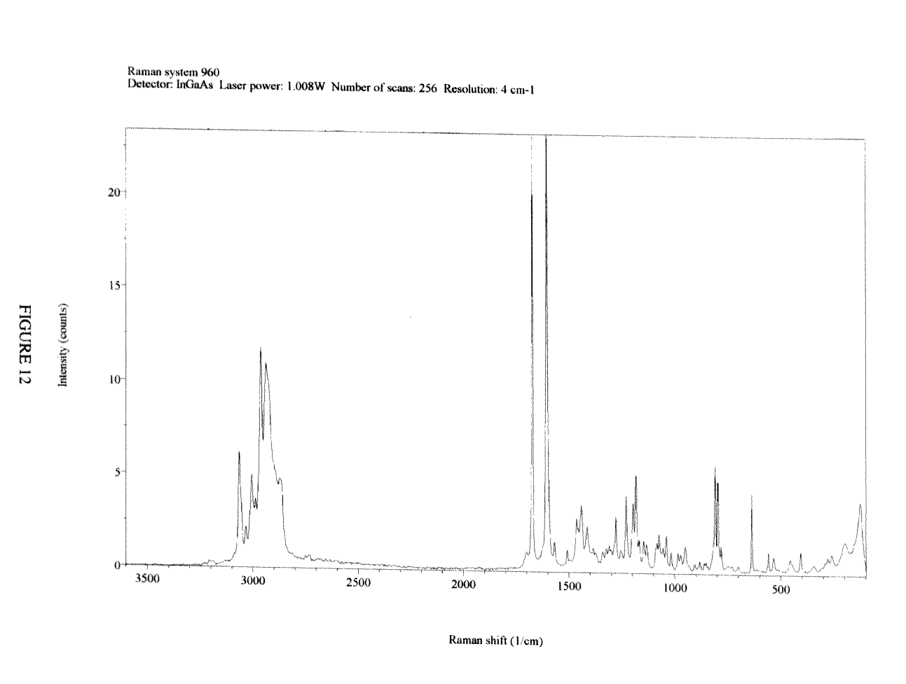

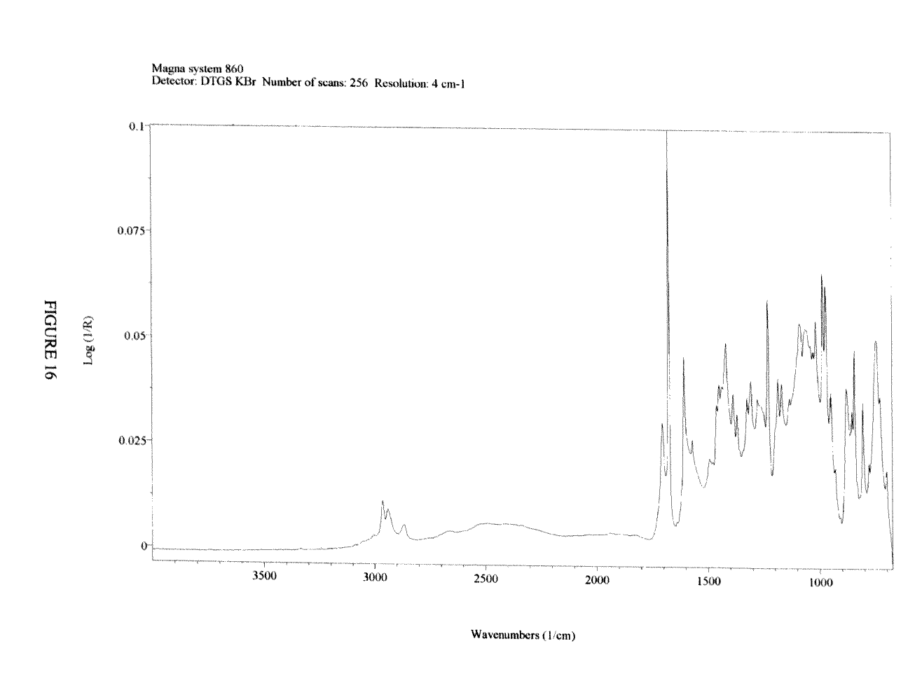

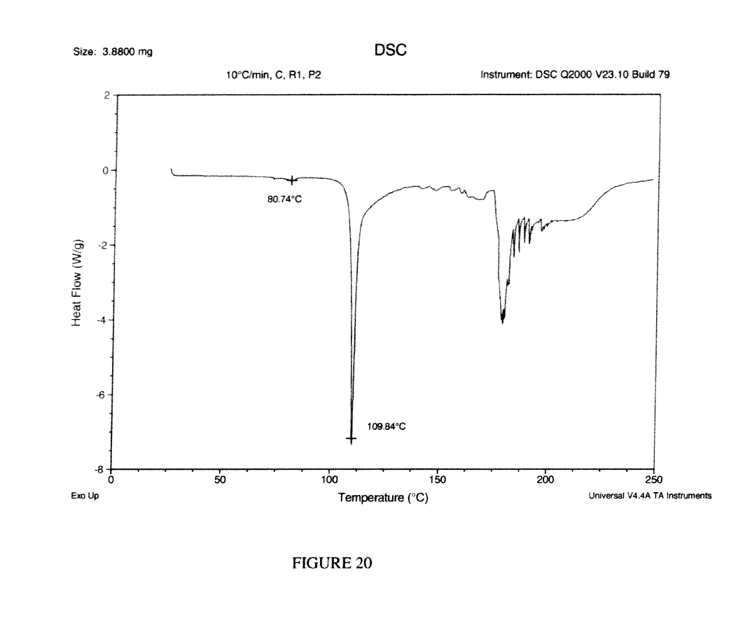

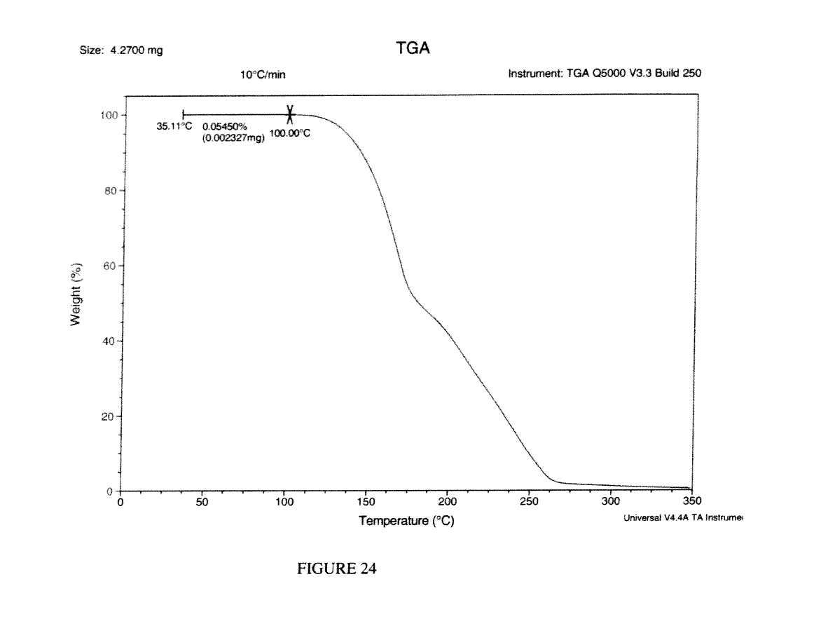

Crystalline racemic eperisone succinate is characterized by an XRPD pattern substantially as shown in FIG. 4, an ¹H-NMR spectrum substantially as shown in FIGS. 8A-8D, a Raman spectra substantially as shown in FIG. 12, an IR spectrum substantially as shown in FIG. 16, a DSC thermogram substantially as shown in FIG. 20, and a TGA profile substantially as shown in FIG. 24. An exemplary listing of representative XRPD peaks of crystalline racemic eperisone succinate according to an embodiment of the invention can be found in Table 7. An exemplary listing of representative NMR data, obtained in CDCl₃, can be found in Table 8.

TABLE 7

| Degrees 2θ | | d spacing Å | | Intensity % (I/Io) |
|---|---|---|---|---|
| 4.94 | ±0.2 | 17.905 | ±0.74 | 15 |
| 9.88 | ±0.2 | 8.951 | ±0.18 | 46 |
| 10.75 | ±0.2 | 8.229 | ±0.15 | 5 |
| 12.10 | ±0.2 | 7.312 | ±0.12 | 13 |
| 13.76 | ±0.2 | 6.436 | ±0.09 | 17 |
| 13.99 | ±0.2 | 6.329 | ±0.09 | 10 |
| 14.21 | ±0.2 | 6.233 | ±0.09 | 7 |
| 14.85 | ±0.2 | 5.968 | ±0.08 | 100 |
| 15.66 | ±0.2 | 5.657 | ±0.07 | 5 |
| 16.82 | ±0.2 | 5.272 | ±0.06 | 11 |
| 17.35 | ±0.2 | 5.111 | ±0.06 | 40 |
| 17.49 | ±0.2 | 5.072 | ±0.06 | 16 |
| 17.69 | ±0.2 | 5.015 | ±0.06 | 5 |
| 18.02 | ±0.2 | 4.923 | ±0.05 | 28 |
| 18.84 | ±0.2 | 4.710 | ±0.05 | 4 |
| 19.16 | ±0.2 | 4.633 | ±0.05 | 18 |
| 19.37 | ±0.2 | 4.582 | ±0.05 | 32 |
| 19.84 | ±0.2 | 4.475 | ±0.04 | 42 |
| 19.98 | ±0.2 | 4.445 | ±0.04 | 45 |
| 22.06 | ±0.2 | 4.029 | ±0.04 | 69 |
| 22.37 | ±0.2 | 3.975 | ±0.04 | 48 |
| 23.32 | ±0.2 | 3.815 | ±0.03 | 21 |
| 23.84 | ±0.2 | 3.733 | ±0.03 | 59 |
| 24.32 | ±0.2 | 3.660 | ±0.03 | 15 |
| 24.87 | ±0.2 | 3.580 | ±0.03 | 23 |
| 25.39 | +0.2 | 3.508 | ±0.03 | 6 |
| 25.99 | ±0.2 | 3.428 | ±0.03 | 3 |
| 26.46 | ±0.2 | 3.369 | ±0.03 | 13 |
| 27.73 | ±0.2 | 3.217 | ±0.02 | 7 |
| 28.67 | +0.2 | 3.114 | ±0.02 | 5 |

TABLE 8

| | peak | | coupling | number |
|---|---|---|---|---|
| | position | | constant | of |
| Protons | (ppm) | multiplicity | (Hz) | protons |
| 2 × CH₃ | 1.23-1.29 | multiplet | — | 6 |
| | | (overlapping | | |
| | | triplets) | | |
| 1 × CH₂ | ~1.58 | broad multiplet | — | 2 |
| 2 × CH₂ | 1.78-1.83 | multiplet | — | 4 |
| succinate CH₂ | 2.48 | singlet | — | 4 |
| CH₂CH₃ | 2.74 | quartet | 8 | 2 |
| 3 × CH₂N | ~2.8-3.2 | broad signal with | — | 4 |
| | | multiplet on top | | |
| | 3.69-3.75 | multiplet | — | 1 |
| CH | 4.23-4.31 | multiplet | — | 1 |
| aromatic | 7.35 | doublet | 8 | 2 |
| aromatic | 7.95 | doublet | 8 | 2 |

Pharmaceutical Compositions and Methods of Treatment and/or Prevention

The novel crystalline forms of eperisone according to various embodiments of the invention possess substantially the same pharmacological activity as racemic eperisone hydrochloride, and are useful for treating and/or preventing the discomfort, muscle spasm, stiffness, or myotonic conditions associated with painful musculoskeletal conditions, such as, for example, low back pain, neck pain, neck-shoulder-arm syndrome, scapulohumeral periarthritis, cervical spondylosis, and other musculoskeletal conditions; spasticity or spastic paralysis of neurological origin due to multiple sclerosis, spinal cord injury, traumatic brain injury, cerebral palsy, stroke or cerebrovascular disorder, spastic spinal paralysis, sequelae of surgical trauma (including, for example, cerebrospinal tumor), amyotrophic lateral sclerosis, spinocerebellar degeneration, spinal vascular disorders, subacute myelo-optico neuropathy (SMON) and other encephalomyelopathies, and other neurological conditions; primary dystonia; secondary dystonia; tension headache; fibromyalgia; chronic fatigue syndrome; muscle cramps; hypertension; and cancer.

The novel crystalline forms of eperisone according to various embodiments of the invention are also useful for treating and/or preventing disorders that arise from altered cell membrane excitability, including, for example, long QT syndrome, Brugada syndrome, heart arrhythmias, malignant hyperthermia, myasthenia, epilepsy, ataxia, migraine, Alzheimer's Disease, Parkinson's Disease, Huntington's Disease, schizophrenia, psychosis, bipolar disorder, hyperekplexia, neuropathic pain and pain associated with nervous system disorders such as, for example, painful diabetic neuropathy, postherpetic neuralgia, trigeminal neuralgia, complex regional pain syndrome I, complex regional pain syndrome II, ischemic neuropathy, phantom limb pain, chemotherapy-induced neuropathy, HIV-related neuropathy, AIDS-related neuropathy, neuropathic back pain, neuropathic neck pain, carpal tunnel syndrome, other forms of nerve entrapment or nerve compression pain, brachial plexus lesions, other peripheral nerve lesions, neuropathic cancer pain, vulvodynia, central neuropathic pain, pain due to multiple sclerosis, post-stroke pain, Parkinson's Disease related central pain, postoperative chronic pain, Guillain-Barre syndrome (GBS), Charcot-Marie-Tooth (CMT) disease, idiopathic peripheral neuropathy, alcoholic neuropathy, other types of neuropathic pain, and other nervous system disorders that have pain as an attendant sign and/or symptom.

The novel crystalline forms of sone according to various embodiments of the invention are also useful for treating and/or preventing non-neuropathic pain of various etiologies, including, by way of example only, inflammatory pain, cancer pain, pain resulting from traumatic injury, post-operative pain, dysmenorrhea, osteoarthritis, rheumatoid arthritis, psoriatic arthritis, gout, tendonitis pain, bursitis pain, sports injury-related pain, sprains, strains, pain of osteoporosis, ankylosing spondylitts, headache, temporomandibular joint pain, interstitial cystitis, myofascial pain syndrome, pain of irritable bowel syndrome, idiopathic chronic pain, and visceral pain.

By use of the term “treating” or “alleviating” it is meant decreasing the symptoms, markers, and/or any negative effects of a condition in any appreciable degree in a patient who currently has the condition, and by “preventing” it is meant preventing entirely or preventing to some extent, such as, for example, by delaying the onset or lessening the degree to which a patient develops the condition.

As discussed, additional embodiments of the invention relate to pharmaceutical compositions comprising a therapeutically effective amount of one or more novel crystalline forms of eperisone according to various embodiments of the invention, and a pharmaceutically acceptable carrier or excipient. The novel crystalline forms of eperisone according to various embodiments of the invention have the same or similar pharmaceutical activity as previously reported for racemic eperisone hydrochloride. Pharmaceutical compositions for the treatment and/or prevention of the enumerated conditions or disorders may contain any amount, for example a therapeutically effective amount, of one or more of the novel crystalline forms of eperisone described herein, as appropriate, e.g. for treatment of a patient with the particular condition or disorder. As a further example, the amount of one or more novel crystalline forms of eperisone in the pharmaceutical compositions may likewise be lower than a therapeutically effective amount, and may, for example, be in the composition in conjunction with another compound or form of eperisone which, when combined, are present in a therapeutically effective amount. A “therapeutically effective amount” as described herein refers to an amount of a therapeutic agent sufficient to treat, alleviate, and/or prevent a condition treatable and/or preventable by administration of a composition of the invention, in any degree. That amount can, for example, be an amount sufficient to exhibit a detectable therapeutic or preventative or ameliorative effect, and can be determined by routine experimentation by those of skill in the art. The effect may include, for example, treatment, alleviation, and/or prevention of the conditions listed herein. The actual amount required, e.g. for treatment of any particular patient, will depend upon a variety of factors including the disorder being treated and/or prevented; its severity; the specific pharmaceutical composition employed; the age, body weight, general health, gender, and diet of the patient; the mode of administration; the time of administration; the route of administration; the rate of excretion of eperisone; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001.

A pharmaceutical composition according to various embodiments of the invention may be any pharmaceutical form which contains one or more novel crystalline forms of eperisone according to various embodiments of the invention. Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition according to various embodiments of the invention, that is one having one or more of the novel crystalline forms of eperisone described herein, a carrier may be chosen that maintains the crystalline form and/or the racemic form. In other words, the carrier, in some embodiments, will not substantially alter the crystalline form and/or the racemic form of the eperisone as described herein. In certain embodiments, the carrier will similarly not be otherwise incompatible with eperisone itself, crystalline salts of eperisone, or racemic crystalline forms of eperisone according to various embodiments of the invention, such as by producing any undesirable biological effect or otherwise interacting in a deleterious manner with any other component(s) of the pharmaceutical composition.

The pharmaceutical compositions according to various embodiments of the invention are optionally formulated in unit dosage form for ease of administration and uniformity of dosage. A “unit dosage form” refers to a physically discrete unit of therapeutic agent appropriate for the patient to be treated. It will be understood, however, that the total daily dosage of the novel crystalline forms of eperisone according to various embodiments of the invention and pharmaceutical compositions thereof will be decided by the attending physician within the scope of sound medical judgment using known methods.

Because the novel crystalline forms of eperisone may be more easily maintained during preparation, solid dosage forms are a preferred form for the pharmaceutical compositions of the invention. Solid dosage forms for oral administration may include, for example, capsules, tablets, pills, powders, and granules. In one exemplary embodiment, the solid dosage form is a tablet. The active ingredient may be contained in a solid dosage form formulation that provides quick release, sustained release, or delayed release after administration to the patient. In such solid dosage forms, the active compound may be mixed with at least one inert, pharmaceutically acceptable carrier, such as, for example, sodium citrate or dicalcium phosphate. The solid dosage form may also include one or more of various additional ingredients, including, for example: a) fillers or extenders such as, for example, starches, lactose, sucrose, glucose, mannitol, and silicic acid; b) binders such as, for example, carboxymethylcellulose, alginates, gelatin, polyvinylpyrrolidinone, sucrose, and acacia; c) humectants such as, for example, glycerol; d) disintegrating agents such as, for example, agar, calcium carbonate, potato or tapioca starch, alginic acid, certain silicates, and sodium carbonate; e) dissolution retarding agents such as, for example, paraffin; f) absorption accelerators such as, for example, quaternary ammonium compounds; g) wetting agents such as, for example, cetyl alcohol and glycerol monostearate; h) absorbents such as, for example, kaolin and bentonite clay; and i) lubricants such as, for example, talc, calcium stearate, magnesium stearate, solid polyethylene glycols, and sodium lauryl sulfate. The solid dosage forms may also comprise buffering agents. They may optionally contain opacifying agents and can also be of a composition that they release the active ingredient(s) only, or preferentially, in a certain part of the intestinal tract, optionally, in a delayed manner. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions according to various embodiments of the invention can also be prepared with coatings and shells such as enteric coatings and other coatings well known in the pharmaceutical formulating art.

The novel crystalline forms of eperisone according to various embodiments of the invention can be, in one exemplary embodiment, administered in a solid micro-encapsulated form with one or more carriers as discussed above. Microencapsulated forms may also be used in soft and hard-filled gelatin capsules with carriers such as lactose or milk sugar as well as high molecular weight polyethylene glycols and the like.

The novel crystalline forms of eperisone according to various embodiments of the invention may also be used in the preparation of non-solid formulations, e.g., injectables and patches, of eperisone. Such non-solid formulations are known in the art. In certain formulations, such as a non-solid formulation, the crystalline salt form may, in certain exemplary embodiments, not be maintained. For example, the crystalline salt form may be dissolved in a liquid carrier. In this case, the novel crystalline forms of eperisone according to various embodiments of the invention may represent intermediate forms of eperisone used in the preparation of the non-solid formulation. The novel crystalline forms of eperisone according to various embodiments of the invention may provide advantages of handling stability and purity to the process of making such formulations.

In addition, the novel crystalline forms of eperisone according to various embodiments of the invention are also useful for administration in combination with other analgesic medication classes, such as strong and weak opioids, NSAIDs, COX-2 inhibitors, acetaminophen, other anti-inflammatories, tricyclic antidepressants, anticonvulsant agents, voltage gated calcium channel blockers, N-type calcium channel blockers, other calcium channel modulators, SNRIs and other monoamine reuptake inhibitors, sodium channel blockers, NK-1 antagonists, NMDA antagonists, AMPA antagonists, other glutamate modulators, GABA modulators, CRMP-2 modulators, TRPV1 agonists, cannabinoids, potassium channel openers, alpha adrenergic agonists, adenosine agonists, nicotinic agonists, p38 MAP kinase inhibitors, corticosteroids, and other analgesic drug classes, and may have a useful dose-sparing effect of lowering the required dosage of the medication used in combination with one or more novel crystalline forms of eperisone according to various embodiments of the invention. The novel crystalline forms of eperisone according to various embodiments of the invention are therefore also useful for treating or preventing complications or side effects arising from usage of other analgesic medications, including problems with opioids such as dependency, constipation, and respiratory depression. Opioid pain medications can either inhibit or excite the CNS, although it is considered that inhibition is more common. Patients with depressed CNS functions may feel varying levels of drowsiness, lightheadedness, euphoria or dysphoria, or confusion. NSAID pain medications can also induce negative side effects, such as gastrointestinal toxicity or bleeding, renal toxicity, and cardiovascular toxicity. Side effects of other analgesic classes can include sedation, dizziness, anticholinergic effects, dependency, hypotension, and various other adverse effects. These analgesic-induced side effects can manifest themselves when the dosage is increased. Decreasing the dosage of an analgesic or changing medications often helps to decrease the rate or severity of these analgesic-induced side effects. It is possible that a therapeutic amount of a novel crystalline form of eperisone according to various embodiments of the invention in combination with a pain agent will reduce the risk of such side effects by reducing the required dosage of the other agent used in combination.

The invention also relates to the treatment and/or prevention of various disorders and/or conditions such as those discussed above, including, for example, pathological muscle contracture, myotonic conditions, spastic paralysis or spasticity caused by various neurologic conditions, and various types of pain and pathological muscle tension. The invention provides a method for treating and/or preventing such disorders and/or conditions by administering to mammals, such as a human, one or more of the novel crystalline forms of *sone as described herein, or a pharmaceutical composition containing the same, in an amount sufficient to treat and/or prevent a condition treatable and/or preventable by administration of a composition of the invention. That amount is the amount sufficient to exhibit any detectable therapeutic and/or preventative or ameliorative effect. The effect may include, for example, treatment and/or prevention of the conditions listed herein. These novel crystalline forms of eperisone and pharmaceutical compositions containing them may, according to various embodiments of the invention, be administered using any amount, any form of pharmaceutical composition, and any route of administration effective, e.g. for treatment and/or prevention, all of which are easily determined by those of skill in the art through routine experimentation. After formulation with an appropriate pharmaceutically acceptable carrier in a desired dosage, as known by those of skill in the art, the pharmaceutical compositions can be administered to humans and other mammals by any known method, such as, for example, orally, rectally, or topically (such as by powders or other solid form-based topical formulations). In certain embodiments, the novel crystalline forms of eperisone according to various embodiments of the invention may be administered at dosage levels ranging from about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than about 0.001 mg/kg or greater than about 50 mg/kg (for example, ranging from about 50 mg/kg to about 100 mg/kg) can also be administered to a subject in certain embodiments of the invention. As discussed above, the amount required for a particular patient will depend upon a variety of factors including the disorder being treated and/or prevented; its severity; the specific pharmaceutical composition employed; the age, body weight, general health, gender, and diet of the patient; the mode of administration; the time of administration; the route of administration; and the rate of excretion of eperisone; the duration of the treatment; any drugs used in combination or coincidental with the specific compound employed; and other such factors well known in the medical arts. And, as also discussed, the pharmaceutical composition of the novel crystalline forms of eperisone as described herein may be administered as a unit dosage form.

Although the present invention herein has been described with reference to various exemplary embodiments, it is to be understood that these embodiments are merely illustrative of the principles and applications of the present invention. Those having skill in the art would recognize that a variety of modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

### EXAMPLES

### Example 1a

### Preparation of Eperisone Free Base

A mixture of 592 mg (2.00 mmol) of racemic eperisone hydrochloride and 10 mL of ethyl acetate was extracted twice with 10-mL portions of a solution of 4% sodium bicarbonate in water and once with 10 mL of water. Shaking dissolved the solids, producing two layers. The ethyl acetate layer was dried over magnesium sulfate and concentrated on a rotary evaporator to give 395 mg (76% yield) of racemic eperisone free base as an oil.

### Example 1b

### Preparation of Eperisone Free Base

A solution of 1.00 g (3.39 mmol) of racemic eperisone hydrochloride in 20 mL of dichloromethane was washed with a solution of 316 mg (3.76 mmol) of sodium bicarbonate in 20 mL of water. The organic layer was washed with one 20-mL portion of water, dried over magnesium sulfate, filtered, and concentrated in vacuo to give an oil. The oil was dissolved in a little dichloromethane and transferred to a tared vial. The vial was left open in the hood under ambient conditions and was placed in a dessicator under diaphragm pump pressure for about 30 min. The resulting colorless oil, containing a small amount of crystalline material, weighed 788 mg. That material was dissolved in about 1 mL of tetrahydrofuran to give a solution containing a little crystalline material. The solution was filtered into a tared vial and the solvent was removed by evaporation in a dessicator under diaphragm pump pressure. The resulting free base was a clear oil that weighed 689 mg (78% yield).

### Example 1c

### Preparation of Eperisone Free Base

A mixture of 3.06 g (10.4 mmol) of racemic eperisone hydrochloride in ?0 mL of ethyl acetate was washed with two 50-mL portions of 4% aqueous sodium bicarbonate and one 50-mL portion of water. The ethyl acetate solution was dried over magnesium sulfate, filtered, and concentrated in vacuo to give 2.13 g (79% yield) of the free base as a colorless oil.

### Example 2a

### Preparation of Crystalline Racemic Eperisone Fumarate

A stirred solution of 105 mg (0.405 mmol) of racemic eperisone free base (Example 1a) in 1 mL of diethyl ether was treated drop wise with a solution of 45 mg (0.39 mmol) of fumaric acid in 1 mL of tetrahydrofuran, resulting in copious precipitation that gave the mixture the appearance of a solid. The mixture was stirred at room temperature for about three days, poured onto a weighing paper, and transferred to a tared vial. The vial was kept in a vacuum oven under mechanical vacuum pump pressure at room temperature for about 24 hours. The remaining solid was found to be 31 mg (20% yield) of racemic eperisone fumarate.

Optical microscopy indicated the solids to be small particles. Analytical data were obtained on the final product: the XRPD pattern was substantially as shown in FIG. 1, and the ¹H-NMR spectrum was substantially as shown in FIGS. 5A-5D.

### Example 2b

### Preparation of Crystalline Racemic Eperisone Fumarate

A stirred solution of 99 mg (0.38 mmol) of racemic eperisone free base (Example 1 a) in 1 mL of diethyl ether was treated drop wise with a solution of 42 mg (0.36 mmol) of fumaric acid in 1 mL of tetrahydrofuran, resulting in precipitation of a solid. The resulting slurry was stirred for about three days and filtered, and the recovered solid was dried in a vacuum oven under mechanical vacuum pump pressure at room temperature for about 24 hours to give 90 mg (63% yield) of crystalline racemic eperisone fumarate.

Optical microscopy indicated the solids to be platy particles. Analytical data were obtained on the final product: the XRPD pattern was as shown in FIG. 1, the ¹H-NMR spectrum was as shown in FIGS. 5A-5D, the Raman spectra as shown in FIG. 9, the IR spectrum was as shown in FIG. 13, the DSC thermogram was as shown in FIG. 17, and the TGA profile was as shown in FIG. 21.

### Example 2c

### Preparation of Crystalline Racemic Eperisone Fumarate

A solution of 628 mg (2.42 mmol) of racemic eperisone free base (Example 1 c) in about 5 mL of diethyl ether was treated with 280 mg (2.41 mmol) of fumaric acid. The resulting slurry was treated with about 2 mL of tetrahydrofuran and stirred at ambient temperature for about 20 minutes. During that time the appearance of the mixture changed from crystalline solid at the bottom of a liquid to a thick, white suspension. An additional 2 mL of diethyl ether were added and the suspension was stirred at ambient temperature for 30 minutes. The suspension was treated with about 2 mL of diethyl ether to render it pourable and was filtered. The filter cake was washed with two 2-mL portions of diethyl ether and dried in a dessicator under diaphragm pump pressure for about 1 hour to give 799 mg (88% yield) of racemic eperisone fumarate as a white solid.

Analytical data were obtained on the final product: the XRPD pattern was substantially as shown in FIG. 1, and the Raman spectrum was substantially as shown in FIG. 9.

### Example 3a

### Preparation of Crystalline Racemic Eperisone Maleate

A stirred solution of 105 mg (0.405 mmol) of racemic eperisone free base (Example 1a) in 1 mL of diethyl ether was treated drop wise with a solution of 47 mg (0.40 mmol) of maleic acid in 1 mL of tetrahydrofuran, resulting in precipitation of a solid. The resulting slurry was stirred at room temperature for about three days and filtered and the recovered solid was dried in a vacuum oven under mechanical vacuum pump pressure at room temperature for about 24 hours to give 105 mg (69% yield) of racemic eperisone maleate.

Optical microscopy indicated the solids to be small particles. Analytical data were obtained on the final product: the XRPD pattern was substantially as shown in FIG. 2, and the ¹H-NMR spectrum was substantially as shown in FIGS. 6A-6E.

### Example 3b

### Preparation of Crystalline Racemic Eperisone Maleate

A stirred solution of 99 mg (0.38 mmol) of racemic eperisone free base (Example 1a) in 1 mL of diethyl ether was treated drop wise with a solution of 40 mg (0.34 mmol) of maleic acid in 1 mL of tetrahydrofuran, resulting in precipitation of solid. The resulting slurry was stirred for about three days and filtered, and the recovered solid was dried in a vacuum oven under mechanical vacuum pump pressure at room temperature for about 24 hours to give 74 mg (52% yield) of racemic eperisone maleate.

Optical microscopy indicated the solids to be platy particles. Analytical data were obtained on the final product: the XRPD pattern was as shown in FIG. 2, the ¹H-NMR spectrum was as shown in FIGS. 6A-6E, the Raman spectra as shown in FIG. 10, the IR spectrum was as shown in FIG. 14, the DSC thermogram was as shown in FIG. 18, and the TGA profile was as shown in FIG. 22.

### Example 3c

### Preparation of Crystalline Racemic Eperisone Maleate

A solution of 669 mg (2.58 mmol) of racemic eperisone free base (Example 1c) in about 4 mL of diethyl ether was treated with 299 mg (2.58 mmol) of maleic acid. The resulting slurry was treated with about 1 mL of tetrahydrofuran and stirred at ambient temperature for about 1.5 hours. During that time the appearance of the mixture changed from crystalline solid at the bottom of a liquid to a thick, white suspension. The suspension was treated with about 2 mL of diethyl ether to render it pourable and was filtered. The filter cake was washed with two 2-mL portions of diethyl ether and dried in a dessicator under diaphragm pump pressure for about 30 minutes to give 921 mg (95% yield) of racemic eperisone maleate as a white solid.

Analytical data were obtained on the final product: the XRPD pattern was substantially as shown in FIG. 2, and the Raman spectrum was substantially as shown in FIG. 10.

### Example 4a

### Preparation of Crystalline Racemic Eperisone Mesylate

A stirred solution of 99 mg (0.38 mmol) of racemic eperisone free base (Example 1a) in 1 mL of diethyl ether was cooled with dry ice and treated, drop wise with occasional agitation, with a solution of 25 μL (0.39 mmol) of methanesulfonic acid in 0.25 mL of diethyl ether. The resulting cloudy mixture was allowed to warm to room temperature, then was agitated on a rotating wheel in the freezer for about one day. Periodically a portion of the mixture was removed and examined with a microscope. Crystallization occurred during that time. Filtration and washing with diethyl ether afforded a solid which was dried in a vacuum oven under mechanical vacuum pump pressure at room temperature for about 24 hours to give 70 mg (52% yield) of racemic eperisone mesylate.

Optical microscopy indicated the solids to be fine needles. Analytical data were obtained on the final product: the XRPD pattern was substantially as shown in FIG. 3, and the ¹H-NMR spectrum was as shown in FIGS. 7A-7F.

### Example 4b

### Preparation of Crystalline Racemic Eperisone Mesylate

A stirred solution of 119 mg (0.459 mmol) of racemic eperisone free base (Example 1a) in 1 mL of diethyl ether was cooled with dry ice and treated, drop wise with occasional agitation, with a solution of 25μL (0.39 mmol) of methanesulfonic acid in 0.25 mL of diethyl ether. The resulting cloudy mixture was agitated on a rotating wheel in the freezer for about seven hours. Crystallization occurred during that time. Filtration and washing with diethyl ether afforded solid which was dried in a vacuum oven under mechanical vacuum pump pressure at room temperature for about 15 hours to give 71 mg (53% yield) of racemic eperisone mesylate.

Optical microscopy indicated the solids to be fine needles. Analytical data were obtained on the final product: the XRPD pattern was as shown in FIG. 3, the Raman spectra as shown in FIG. 11, the IR spectrum was as shown in FIG. 15, the DSC thermogram was as shown in FIG. 19, and the TGA profile was as shown in FIG. 23.

### Example 4c

### Preparation of Crystalline Racemic Eperisone Mesylate

Racemic eperisone free base (Example 1b) (689 mg, 2.66 mmol) was dissolved in about 1.5 mL of tetrahydrofuran, filtered through glass fiber paper, and treated with a solution of 254 mg (2.64 mmol) of methanesulfonic acid in about 1 mL of tetrahydrofuran. The resulting solution was treated with hexanes drop wise until just before the cloud point (about 0.75 mL of hexanes) and placed in the refrigerator. A liquid layer separated and the resulting two-phase mixture was placed in the freezer. The lower layer crystallized. After a couple hours in the freezer the crystalline material was removed by filtration, washed with about 0.5 mL of hexanes, and dried for about 1 hour in a dessicator under diaphragm pump pressure to give 723 mg (76% yield) of racemic eperisone mesylate as a somewhat sticky, white, crystalline solid.

Analytical data were obtained on the final product: the XRPD pattern was substantially as shown in FIG. 3, and the Raman spectrum was substantially as shown in FIG. 11.

### Example 5a

### Preparation of Crystalline Racemic Eperisone Succinate

A stirred solution of 105 mg (0.405 mmol) of racemic eperisone free base (Example 1a) in 1 mL of diethyl ether was treated drop wise with a solution of 48 mg (0.41 mmol) of succinic acid in 1 mL of tetrahydrofuran. The resulting solution was stirred at room temperature for about three days, during which time a solid precipitated. Filtration afforded a solid which was dried in a vacuum oven under mechanical vacuum pump pressure at room temperature for about 24 hours to give 57 mg (37% yield) of racemic eperisone succinate.

Optical microscopy indicated the solids to be small particles. Analytical data were obtained on the final product: the XRPD pattern was substantially as shown in FIG. 4, and the ¹H-NMR spectrum was substantially as shown in FIGS. 8A-8D.

### Example 5b

### Preparation of Crystalline Racemic Eperisone Succinate

A stirred solution of 99 mg (0.38 mmol) of racemic eperisone free base (Example la) in 1 mL of diethyl ether was treated drop wise with a solution of 49 mg (0.41 mmol) of succinic acid in 1 mL of tetrahydrofuran. The resulting solution was stirred for about three days, during which time a solid precipitated. The solid was recovered by filtration and dried in a vacuum oven under mechanical vacuum pump pressure at room temperature for about 24 hours to give 74 mg (52% yield) of racemic eperisone succinate.

Optical microscopy indicated the solids to be small particles. Analytical data were obtained on the final product: the XRPD pattern as a shown in FIG. 4, the ¹H-NMR spectrum was as shown in FIGS. 8A-8D, the Raman spectra as shown in FIG. 12, the IR spectrum was as shown in FIG. 16, the DSC thermogram was as shown in FIG. 20, and the TGA profile was as shown in FIG. 24.

### Example 5c

### Preparation of Crystalline Racemic Eperisone Succinate

A solution of 749 mg (2.89 mmol) of racemic eperisone free base (Example 1c) in about 6 mL of diethyl ether was treated with 340 mg (2.88 mmol) of succinic acid. The resulting slurry was treated with about 2 mL of tetrahydrofuran and stirred at ambient temperature for about 40 minutes. During that time the appearance of the mixture changed from crystalline solid at the bottom of a liquid to a thick, white suspension. An additional 2 mL of diethyl ether were added and the suspension was stirred at ambient temperature for 35 minutes. The suspension was treated with about 2 mL of diethyl ether to render it pourable and was filtered. The filter cake was washed with two 2-mL portions of diethyl ether and dried in a dessicator under diaphragm pump pressure for about 1 hour to give 855 mg (79% yield) of racemic eperisone succinate as a white solid.

Analytical data were obtained on the final product: the XRPD pattern was substantially as shown in FIG. 4, and the Raman spectrum was substantially as shown in FIG. 12.

### Example 6

### Solubility Determinations

The solubilities of the crystalline forms of racemic eperisone were determined as follows. Each experiment was conducted in a one-dram vial by adding weighed amounts of solid salt in portions to a weighed amount of HPLC-grade water until a slurry was obtained. In the case of the mesylate salt, a slurry was not obtained; solid addition was stopped while a solution was still present. Each vial was capped and placed on a rotating wheel for 46.5 hours at ambient temperature. Each vial was removed from the wheel. The ambient temperature at the time of removal was about 19° C. The contents of each vial were vacuum filtered through Whatman Grade 1 filter paper. Solids adhering to the inside of the vial were not recovered. Each filtrate was weighed, dried under diaphragm pump pressure over phosphorus pentoxide, and the resulting solid residue was weighed. Each filter cake was allowed to dry in the air overnight and was weighed. Experimental details are shown in Table 9.

TABLE 9

| Salt | Fumarate | HCl | Maleate | Mesylate | Succinate |
|---|---|---|---|---|---|
| Wt.^{a} Solid Added | 52.6 | 182.0 | 68.2 | 203.3 | 175.3 |
| Wt. Water Added | 1022.5 | 519.9 | 1021.1 | 518.4 | 1036.1 |
| Wt. Filtrate | 599.7 | 329.2 | 671.5 | — | 732.5 |
| Wt. Filtrate After | 9.9 | 57.3 | 13.6 | — | 75.4 |
| Drying | | | | | |
| Wt. Filter Cake | 14.5 | 43.0 | 33.7 | — | 40.8 |

^{a}Wt. = weight in milligrams.

The calculated solubilities are shown in Table 10.

TABLE 10

| Salt | Solubility (mg/mL) |
|---|---|
| Fumarate | 16.8 |
| Hydrochloride | 210.7 |
| Maleate | 20.7 |
| Mesylate | >392.2 |
| Succinate | 114.7 |

As can be seen in Table 10, a wide range of solubilities exists among the forms, from the novel racemic eperisone mesylate form (most soluble) to the novel racemic eperisone fumarate form (least soluble). Such variability in solubility would be expected to offer benefits, such as, for example, improved and/or alternative formulation options, increased or decreased bioavailability, as needed, as well as others.

### Example 7a

### Recrystallization of Racemic Eperisone Fumarate Salt

A sample of product from Example 2c was placed in a one-dram vial containing a stir bar. Some dichloromethane was added and the salt dissolved. The vial was heated on a hot plate with stirring until gentle reflux was obtained. Hexanes were added drop wise to maintain a constant volume. When the solution became cloudy, dichloromethane was added drop wise until it cleared. Stirring was stopped, the hot plate was turned off, and the vial was capped. The vial was left on the hot plate to cool slowly as the hot plate cooled to room temperature. Crystals formed in the vial. The mixture was placed in the refrigerator overnight and vacuum filtered to give crystals.

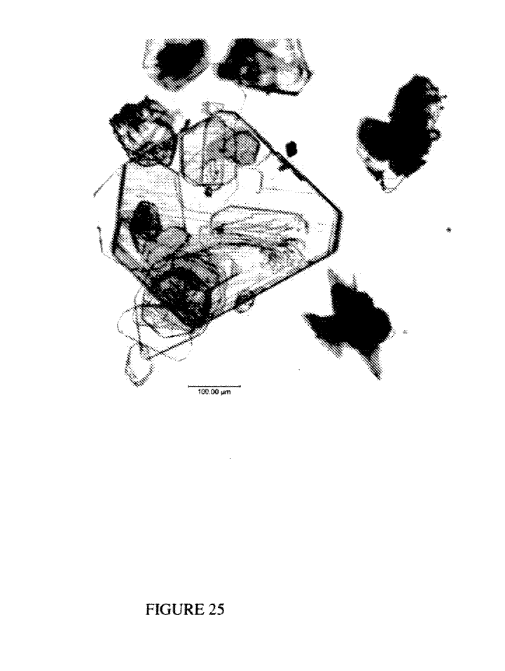

A photo micrograph was obtained of a sample of those crystals, as seen in FIG. 25.

### Example 7b

### Recrystallization of Racemic Eperisone Maleate Salt

A sample of product from Example 3c was placed in a one-dram vial containing a stir bar. Some dichloromethane was added and the salt dissolved. The vial was heated on a hot plate with stirring until gentle reflux was obtained. Hexanes were added drop wise to maintain a constant volume. When the solution became cloudy, dichloromethane was added drop wise until it cleared. Stirring was stopped, the hot plate was turned off, and the vial was capped. The vial was left on the hot plate to cool slowly as the hot plate cooled to room temperature. Crystals formed in the vial. The mother liquor was decanted, leaving crystals.

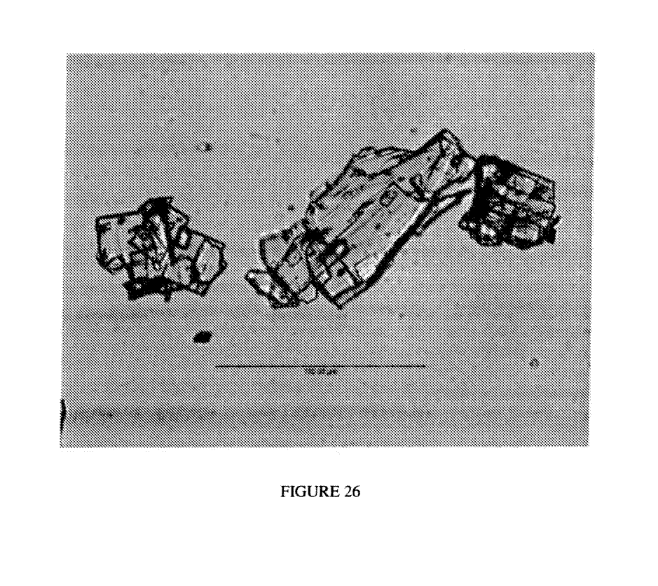

A photo micrograph was obtained of a sample of those crystals, as seen in FIG. 26.

### Example 7c

### Recrystallization of Racemic Eperisone Mesylate Salt

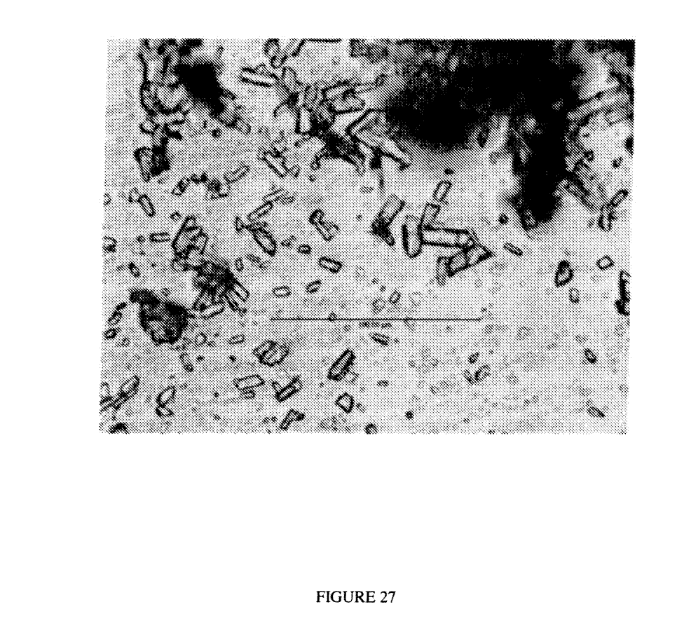

Attempted crystallization of the product from Example 4c afforded racemic eperisone mesylate salt as an oil. Accordingly, a photo micrograph was obtained of the crystals produced in Example 4c, as seen in FIG. 27.

### Example 7d

### Recrystallization of Racemic Eperisone Succinate Salt

A sample of product from Example 5c was placed in a one-dram vial containing a stir bar. Some dichloromethane was added and the salt dissolved. The vial was heated on a hot plate with stirring until gentle reflux was obtained. Hexanes were added drop wise to maintain a constant volume. When the solution became cloudy, dichloromethane was added drop wise until it cleared. Stirring was stopped, the hot plate was turned off, and the vial was capped. The vial was left on the hot plate to cool slowly as the hot plate cooled to room temperature. Crystals formed in the vial. The mixture was placed in the refrigerator for a couple hours. The mother liquor was decanted, leaving crystals.

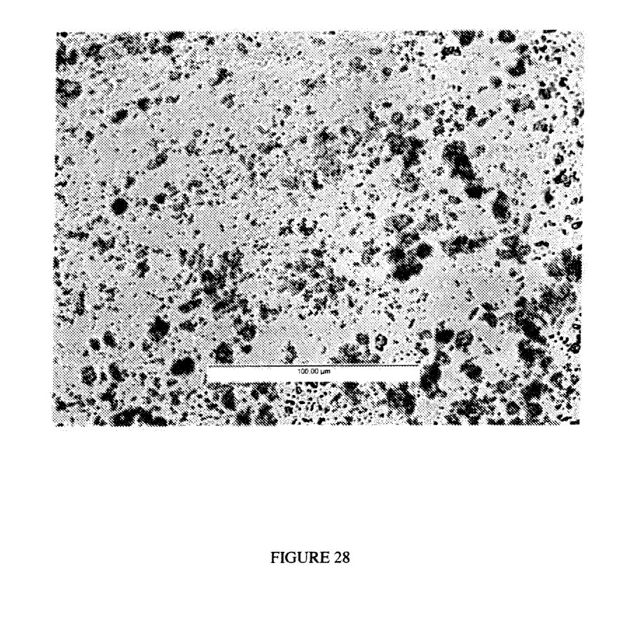

A photo micrograph was obtained of a sample of those crystals, as seen in FIG. 28.

### Example 7e

### Recrystallization of Racemic Eperisone Hydrochloride Salt

A sample of racemic eperisone hydrochloride salt was placed in a one-dram vial containing a stir bar. Some dichloromethane was added and the salt dissolved. The vial was heated on a hot plate with stirring until gentle reflux was obtained. Hexanes were added drop wise to maintain a constant volume. When the solution became cloudy, dichloromethane was added drop wise until it cleared. Stirring was stopped, the hot plate was turned off, and the vial was capped. The vial was left on the hot plate to cool slowly as the hot plate cooled to room temperature. After a couple hours crystals had formed in the vial. The mother liquor was removed by pipette, leaving crystals in the vial.

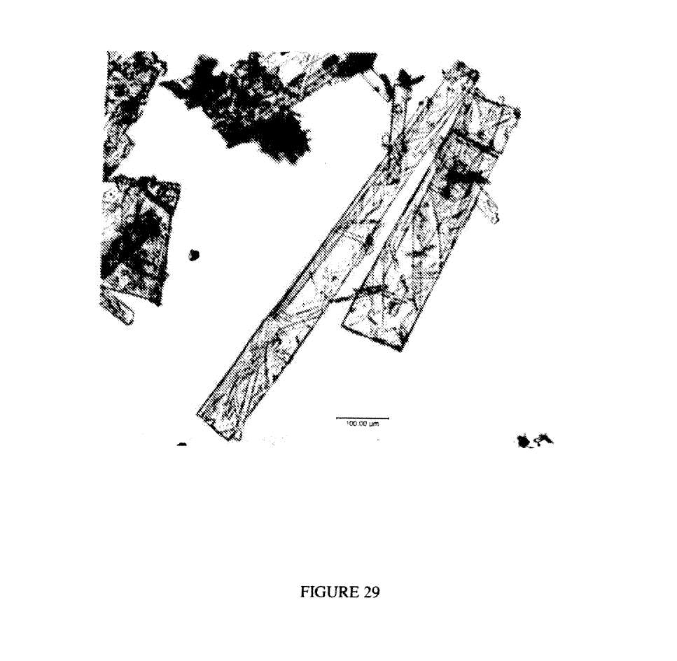

A photo micrograph was obtained of a sample of those crystals, as seen in FIG. 29.

In general, there are several forms of crystal habits that crystals may exhibit. Some of the common known groups of crystal habits include planar (plate-like), acicular (needle-shaped) and equant (particles of roughly similar length, width and thickness, including both cubical and spherical particles). Crystals having the same polymorphic structure, i.e. the same unique arrangement of molecules inside the crystal, may still exhibit different crystal habits. It is known that the crystal habit and morphology, the external structure of a crystal, plays a significant role in flowability, packing, compaction, suspension stability, dissolution, tablet compressibility, mechanical strength, and sedimentation characteristics of solid pharmaceuticals. It is therefore desirable to identify a range of habits of eperisone in order to optimize the manufacturing properties of the final dosage form. As can be seen in FIGS. 25-29, the aspect ratio of the crystals of the various novel forms of racemic eperisone varies significantly among the forms. Further, the shape of the crystals of each form is also observed to be rather dissimilar. Such variability in the crystal size and shape of the novel forms of eperisone may be expected to offer benefits, such as, for example, the ability to improve handling and/or filtering properties by selecting one crystal form of racemic eperisone over another.

Crystal size and particle size distribution is also known to vary significantly and to have an impact on many pharmaceutical factors, including dissolution, absorption rates, content uniformity, compressibility, and flowability. Smaller crystals have a higher surface area to volume ratio, and typically have faster dissolution rates than larger crystals; efforts to reduce crystal or particle size, including micronization, nanocrystallization, and other technologies, are commonly used to increase dissolution rates and bioavailability. Given particle size's impact on bioavailability, the safety profile of a drug can also be improved by dosing with more consistent and defined particle sizes, allowing for greater reliability in the dosing of the drug necessary to achieve a desired result. Content uniformity is a measure of the amount of API contained in dosage forms; high content uniformity ensures that a consistent amount of API is delivered with each dose. APIs with a wide particle size distribution may have a negative impact on content uniformity, with a resultant variation in actual amount of API delivered with each dose. Crystal size and distribution is also known to affect manufacturing properties, including compressibility and flowability. Various efforts have been employed to ensure a particle size distribution in a narrow reproducible range, many of which are labor or energy intensive, or result in significant loss of API, including spray drying, multi-stage milling techniques, and the combination of extrusion with spheronising. As can be seen in FIGS. 25-29, the crystal size and particle size distribution of the crystals of the various novel forms of racemic eperisone varies significantly among the forms. The observed variability in the crystal size and particle size distribution of the novel forms of eperisone may be expected to offer benefits, such as, for example, the ability to improve manufacturing or dosing properties by selecting one crystal form of racemic eperisone over another.

1. A crystalline salt of (2RS)-1-(4-ethylphenyl)-2-methyl-3-piperidin-1-ylpropan-1-one, chosen from a crystalline racemic fumarate salt, a crystalline racemic maleate salt, a crystalline racemic mesylate salt, and a crystalline racemic succinate salt.

2-4. (canceled)

5. A crystalline racemic fumarate salt of (2RS)-1-(4-ethylphenyl)-2-methyl-3-piperidin-1-yl-propan-1-one having substantially the same XRPD pattern as shown in FIG. 1.

6. A crystalline racemic maleate salt of (2RS)-1-(4-ethylphenyl)-2-methyl-3-piperidin-1-yl-propan-1-one having substantially the same XRPD pattern as shown in FIG. 2.

7. A crystalline racemic mesylate salt of (2RS)-1-(4-ethylphenyl)-2-methyl-3-piperidin-1-yl-propan-1-one having substantially the same XRPD pattern as shown in FIG. 3.

8. A crystalline racemic succinate salt of (2RS)-1-(4-ethylphenyl)-2-methyl-3-piperidin-1-yl-propan-1-one having substantially the same XRPD pattern as shown in FIG. 4.

9. A pharmaceutical composition comprising at least one of the crystalline racemic salts of (2RS)-1-(4-ethylphenyl)-2-methyl-3-piperidin-1-yl-propan-1-one according to claim 1.

10-12. (canceled)

13. A pharmaceutical composition comprising the crystalline racemic fumarate salt of (2RS)-1-(4-ethylphenyl)-2-methyl-3-piperidin-1-yl-propan-1-one according to claim 5.

14. A pharmaceutical composition comprising the crystalline racemic maleate salt of (2RS)-1-(4-ethylphenyl)-2-methyl-3-piperidin-1-yl-propan-1-one according to claim 6.

15. A pharmaceutical composition comprising the crystalline racemic mesylate salt of (2RS)-1-(4-ethylphenyl)-2-methyl-3-piperidin-1-yl-propan-1-one according to claim 7.

16. A pharmaceutical composition comprising the crystalline racemic succinate salt of (2RS)-1-(4-ethylphenyl)-2-methyl-3-piperidin-1-yl-propan-1-one according to claim 8.

17. A method of treating and/or preventing any of the following conditions, discomfort, muscle spasm, stiffness, or myotonic conditions associated with musculoskeletal conditions; spasticity or spastic paralysis of neurological origin; dystonia; headache; fibromyalgia; chronic fatigue syndrome; muscle cramps; pain of various etiologies; and disorders that arise from altered cell membrane excitability, said method comprising administering a pharmaceutical composition according to claim 9 to a patient in need thereof.
